(19) United States
(12) Patent Application Publication
(10) Pub. No.: US 2007/0265325 A1
(43) Pub. Date: Nov. 15, 2007

(54) Nitro Indazole Derivatives

(75) Inventors: Vijayaratnam Santhakumar, Quebec (CA); Miroslaw Tomaszewski, Quebec (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/718,739

(22) PCT Filed: Nov. 7, 2005
(86) PCT No.: PCT/SE05/01668

§ 371 Date: May 7, 2007

(30) Foreign Application Priority Data
Nov. 11, 2004 (SE) 0402763-7

(51) Int. Cl. A61K 31/416; A61P 1/00; A61P 25/12; A61P 25/14; A61P 25/22; A61P 25/28; A61P 35/00; A61P 43/00; A61P 9/00; C07D 231/54

(52) U.S. Cl. 514/403; 548/362.5

(57) ABSTRACT

Compounds of Formula I or pharmaceutically acceptable salts thereof. 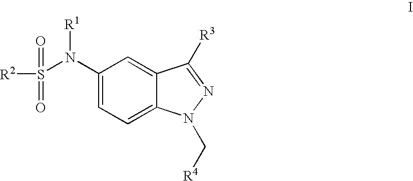
wherein R¹, R², R³ and R⁴ are as defined in the specification as well as salts and pharmaceutical compositions including the compounds are prepared. They are useful in therapy, in particular in the management of pain.

### BACKGROUND OF THE INVENTION

1. Field of the Invention

The invention is related to therapeutic compounds which are CB₁ receptor ligands, pharmaceutical compositions containg these compounds, manufacturing processes thereof and uses thereof, and more particularly to compounds that are CB₁ receptor agonists. More particularly, the present invention is related to compounds that may be effective in treating pain, cancer, multiple sclerosis, Parkinson's disease, Huntington's chorea, Alzheimer's disease, anxiety disorders, gastrointestinal disorders and cardiavascular disorders.

2. Discussion of Relevant Technology

Pain management has been an important field of study for many years. It has been well known that cannabinoid receptor (e.g., CB₁ receptor, CB₂ receptor) ligands including agonists, antagonists and inverse agonists produce relief of pain in a variety of animal models by interacting with CB₁ and/or CB₂ receptors. Generally, CB₁ receptors are located predominately in the central nervous system, whereas CB₂ receptors are located primarily in the periphery and are primarily restricted to the cells and tissues derived from the immune system.

While CB₁ receptor agonists, such as Δ⁹-tetrahydrocannabinol (Δ⁹-THC) and anadamide, are useful in anti-nociception models in animals, they tend to exert undesired CNS side-effects, e.g., psychoactive side effects, the abuse potential, drug dependence and tolerance, etc. These undesired side effects are known to be mediated by the CB₁ receptors located in CNS. There are lines of evidence, however, suggesting that CB₁ agonists acting at peripheral sites or with limited CNS exposure can manage pain in humans or animals with much improved overall in vivo profile.

Therefore, there is a need for new CB₁ receptor ligands such as agonists, antagonists or inverse agonists that are useful in managing pain or treating other related symptoms or diseases with reduced or minimal undesirable CNS side-effects.

### DISCLOSURE OF THE INVENTION

The present invention provides CB₁ receptor ligands which are useful in treating pain and other related symptoms or diseases.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

“CB₁/CB₂ receptors” means CB₁ and/or CB₂ receptors.

The term “Cₘ₋ₙ” or “Cₘ₋ₙ group” used alone or as a prefix, refers to any group having m to n carbon atoms, and having 0 to n multivalent heteroatoms selected from O, S, N and P, wherein m and n are 0 or positive integers, and n>m. For example, “C₁₋₆” would refer to a chemical group having 1 to 6 carbon atoms, and having 0 to 6 multivalent heteroatoms selected from O, S, N and P.

The term “hydrocarbon” used alone or as a suffix or prefix, refers to any structure comprising only carbon and hydrogen atoms up to 14 carbon atoms. The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms. Unless otherwise specified, “alkyl” general includes both saturated alkyl and unsaturated alkyl.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “allkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms, wherein the radical is located on a carbon of the aromatic ring.

The term “non-aromatic group” or “non-aromatic” used alone, as suffix or as prefix, refers to a chemical group or radical that does not containing a ring having aromatic character (e.g., 4n+2 delocalized electrons).

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alone or as a suffix or prefix, refers to a radical formed as a result of replacing one or more carbon atom of an alkyl with one or more heteroatoms selected from N, O, P and S.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen from a carbon of a ring of the heterocycle.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character, wherein the radical of the heterocyclyl is located on a carbon of an aromatic ring of the heterocyclyl.

The term “heterocylcoalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O, and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₁₂hydrocarbyl For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazohnyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein —R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “aryloxy” used alone or as suffix or prefix, refers to radicals of the general formula —O—Ar′, wherein —Ar′ is an aryl.

The term “heteroaryloxy” used alone or as suffix or prefix, refers to radicals of the general formula —O—Ar′, wherein —Ar′ is a heteroaryl.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means —C(═O)—R, wherein —R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

Halogen includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least two atoms therebetween.

“Link,” “linked,” or “linking,” unless otherwise specified, means covalently linked or bonded.

When a first group, structure, or atom is “directly connected” to a second group, structure or atom, at least one atom of the first group, structure or atom forms a chemical bond with at least one atom of the second group, structure or atom.

“Saturated carbon” means a carbon atom in a structure, molecule or group wherein all the bonds connected to this carbon atom are single bond. In other words, there is no double or triple bonds connected to this carbon atom and this carbon atom generally adopts an sp³ atomic orbital hybridization.

“Unsaturated carbon” means a carbon atom in a structure, molecule or group wherein at least one bond connected to this carbon atom is not a single bond. In other words, there is at least one double or triple bond connected to this carbon atom and this carbon atom generally adopts a sp or sp² atomic orbital hybridization.

In one aspect, the invention provides a compound of Formula I, pharmaceutically acceptable salts thereof, diastereomers, enantiomers, or mixtures thereof: 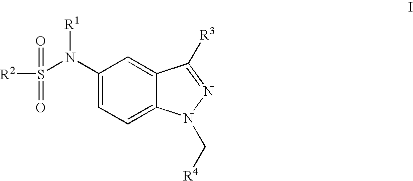
wherein

R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;

R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄-8cycloalkenyl, C₄₋₈cycloalkenyl-C₁₋₆alkyl, C₃₋₆heterocyclyl, and C₃₋₆heterocycloalkyl, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl used in defining R² is optionally substituted with one or more groups selected from C₁₋₆alkyl, halogen, amino and C₁₋₆alkoxy,

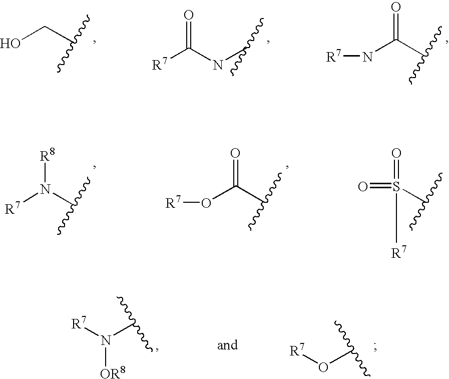

each of R⁷ and R⁸ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆group that together with another divalent group selected from R⁷ and R⁸ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and divalent C₁₋₆group used in defining R⁷ and R⁸ is optionally substituted by one or more groups selected from halogen, cyano, nitro, methoxy, ethoxy, methyl, ethyl, hydroxy, and —NR⁵R⁶;

R³ is selected from hydrogen, halogen, amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀-cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from C₁₋₄alkyl, C₂₋₄alkenyl, C₂₋₄alkynyl, halogen, C₁₋₄alkoxy, amino, nitro, cyano, oxo, hydroxy, C₁₋₆cycloalkyl-C₁₋₆alkyl, C₃₋₆heterocyclyl, C₃₋₆heterocyclyl-C₁₋₆alkyl, and —NR⁵R⁶

wherein R⁵ and R⁶ are independently selected from —H, C₁₋₈alkyl, C₂₋₈alkenyl, C₂₋₈alkynyl, and a divalent C₁₋₈group that together with another divalent R⁵ or R⁶ may form a ring or a portion of a ring; and

R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)—; wherein said C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

The compounds of the present invention also include those of Formula I, wherein

R¹ is selected from from —H and C₁₋₆alkyl;

R² is selected from C₁₋₈alkyl, C₂₋₈alkenyl, C₂₋₈alkynyl, C₃₋₈cycloalkyl, C₃₋₈heterocyclyl and C₃₋₈heterocycloalkyl; wherein said C₁₋₈alkyl, C₂₋₈alkenyl, C₂₋₈alkynyl, C₃₋₈cycloalkyl, C₃₋₈heterocyclyl and C₃₋₈heterocycloalkyl used in defining R² is optionally substituted with one or more groups selected from C₁₋₈alkyl, halogen, amino, hydroxy and C₁₋₆alkoxy;

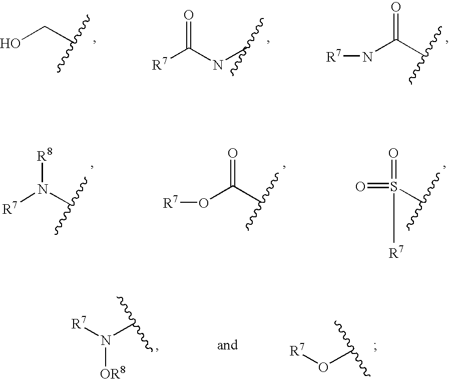

each of R⁷ and R⁸ is independently selected from —H, C₁₋₈alkyl, C₁₋₈alkoxy, C₂₋₈alkenyl, C₃₋₈cycloalkyl, C₃₋₈cycloalkyl-C₁₋₆alkyl, C₃₋₈heterocyclyl and C₃₋₈heterocylcyl-C₁₋₆alkyl; wherein said C₁₋₈alkyl, C₂₋₈alkenyl, C₃₋₈cycloalkyl, C₃₋₈cycloalkyl-C₁₋₆alkyl, C₃₋₈heterocyclyl and C₃₋₈heterocylcyl-C₁₋₆alkyl used in defining R⁷ and R⁸ are optionally substituted by one or more groups selected from halogen, cyano, nitro, methoxy, ethoxy, methyl, ethyl, hydroxy and —NR⁵R⁶;

R³ is selected from hydrogen, halogen, amino, C₁₋₈alkyl, C₂₋₈alkenyl, C₂₋₈alkynyl, C₃₋₈cycloalkyl, C₄₋₈cycloalkenyl, C₃₋₈heteroaryl, R⁵R⁶N—, C₃₋₈cycloalkyl-C₁₋₄alkyl, C₄₋₈cycloalkenyl-C₁₋₄alkyl, phenyl, phenyl-C₁₋₄alkyl, C₃₋₈heterocyclyl or C₃₋₈heterocyclyl-C₁₋₄alkyl; wherein said amino, C₁₋₈alkyl, C₂₋₈alkenyl, C₂₋₈alkynyl, C₃₋₈cycloalkyl, C₄₋₈cycloalkenyl, C₃₋₈heteroaryl, R⁵R⁶N—, C₃₋₈cycloalkyl-C₁₋₄alkyl, C₄₋₈cycloalkenyl-C₁₋₄alkyl, phenyl, phenyl-C₁₋₄alkyl, C₃₋₈heterocyclyl or C₃₋₈heterocyclyl-C₁₋₄alkyl used in defining R³ is optionally substituted by one or more groups selected from halogen, cyano, nitro, C₁₋₄alkoxy, C₁₋₄alkyl, C₂₋₄alkenyl, C₂₋₄alkynyl, hydroxy, and —NR⁵R⁶;

wherein R⁵ and R⁶ are independently selected from from —H, C₁₋₆alkyl, and C₂₋₆alkenyl; and

R⁴ is selected from C₁₋₈alkyl, C₂₋₈alkenyl, C₂₋₈alkynyl, C₃₋₈cycloalkyl, C₃₋₈cycloalkyl-C₁₋₄alkyl, C₄₋₈cycloalkenyl, C₆₋₁₀aryl, phenyl, alkyl, phenyl-C₁₋₄alkyl, C₃₋₈heterocyclyl or C₃₋₈heterocyclyl-C₁₋₄alkyl; wherein said C₁₋₈alkyl, C₂₋₈alkenyl, C₂₋₈alkynyl, C₃₋₈cycloalkyl, C₃₋₈cycloalkyl-C₁₋₄alkyl, C₄₋₈cycloalkenyl, C₆₋₁₀aryl, phenyl, alkyl, phenyl-C₁₋₄alkyl, C₃₋₈heterocyclyl or C₃₋₈heterocyclyl-C₁₋₄alkyl used in defining R⁴ is optionally substituted by one or more groups selected from C₁₋₄alkyl, C₁₋₄alkoxy, halogen, cyano, amino, nitro, oxo, hydroxy, and —NR⁵R⁶.

The compounds of the present invention also include those of Formula I, wherein

R¹ is selected from —H and C₁₋₄alkyl;

R² is selected from C₁₋₆alkyl, C₂₋₆alkenyl, C₃₋₆cycloalkyl, C₃₋₆cycloalkenyl, C₃₋₆heterocyclyl, C₃-6heterocycloalkyl; wherein said C₁₋₆alkyl, C₂₋₆alkenyl, C₃₋₆cycloalkyl, C₃₋₆cycloalkenyl, C₃-6heterocyclyl, C₃₋₆heterocycloalkyl used in defining R² is optionally substituted with one or more C₁₋₆alkyl, C₁₋₆alkoxy,

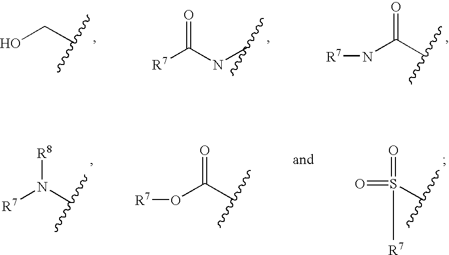

wherein each of R⁷ and R⁸ is independently selected from —H, C₁₋₆alkyl, C₂₋₆alkenyl, C₂₋₆alkynyl, C₁₋₆alkoxy and C₃₋₆cycloalkyl;

R³ is selected from hydrogen, halogen, amino, C₁₋₆alkyl, C₂₋₆alkenyl, C₂₋₆alkynyl, C₃₋₆cycloalkyl, C₃₋₆cycloalkyl-C₁₋₄alkyl, C₃₋₆heterocyclyl or C₃₋₆heterocyclyl-C₁₋₄alkyl wherein said amino, C₁₋₆alkyl, C₂₋₆alkenyl, C₃₋₆cycloalkyl, C₃₋₆cycloalkyl-C₁₋₄alkyl, C₃₋₆heterocyclyl or C₃₋₆heterocyclyl-C₁₋₄alkyl used in defining R³ is optionally substituted by one or more groups selected from Cl, F, methoxy, ethoxy, methyl, ethyl and hydroxy;

R⁵ and R⁶ are independently selected from —H and C₁₋₃alkyl; and

R⁴ is selected from C₁₋₆alkyl, C₂₋₆alkenyl, C₂₋₆alkynyl, phenyl, alkyl, C₃₋₆cycloalkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, wherein said phenyl, alkyl, phenyl-C₁₋₄alkyl, C₃₋₆cycloalkyl-C₁₋₄alkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, used in defining R⁴ is optionally substituted by one or more groups selected from C₁₋₄alkyl, C₁₋₄alkoxy, halogen, amino, cyano, oxo, hydroxy, and —NR⁵R⁶.

The compounds of the present invention also include those of Formula I, wherein

R¹ is selected from —H and C₁₋₃alkyl;

R² is methyl, ethyl, propyl, t-butyl, n-butyl, phenyl, benzyl, and thienyl, wherein said phenyl, benzyl, and thienyl used in defining R² is optionally substituted with one or more methyl, ethyl, propyl, hydroxy, methoxy, ethoxy,

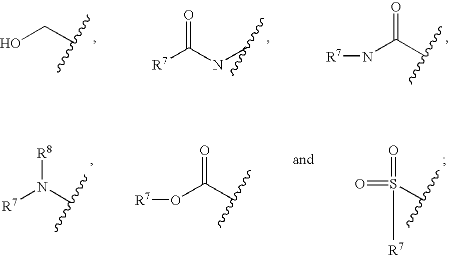

each of R⁷ and R⁸ is independently is selected from —H, methyl, ethyl, propyl, butyl, hydroxy, methoxy;

R³ is selected from hydrogen and Cl; and

R⁴ is selected from C₁₋₄alkyl, C₂₋₄alkenyl, C₂₋₄alkynyl, phenyl, cyclohexyl, cyclopentyl, cyclobutyl, cyclopropyl, cyclohexyl, cyclopentyl, and benzyl; wherein said phenyl, cyclohexyl, cyclopentyl, cyclobutyl, cyclopropyl, cyclohexyl, cyclopentyl, and benzyl used in defining R⁴ is optionally substituted by one or more groups selected from fluorine, chlorine, methoxy, ethoxy, methyl, butyl, propyl, ethyl and hydroxy.

In another aspect, the invention also provides a compound of Formula II, pharmaceutically acceptable salts thereof, diastereomers, enantiomers, or mixtures thereof: 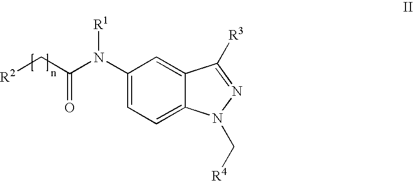
wherein

R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;

R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl 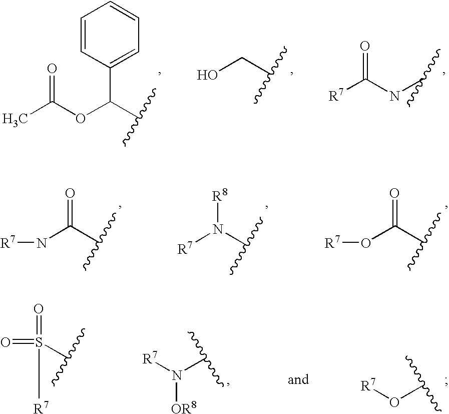
wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl 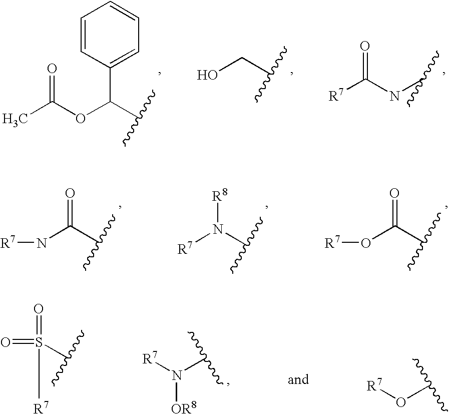
used in defining R² is optionally substituted with one or more groups selected from C₁₋₆alkyl, halogen, amino and C₁₋₆alkoxy,

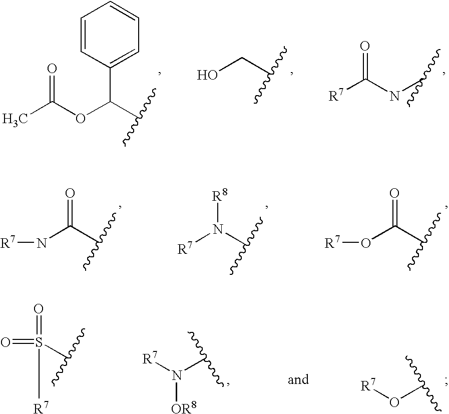

each of R⁷ and R⁸ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆group that together with another divalent group selected from R⁸ and R⁹ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, or divalent C₁₋₆group is optionally substituted by one or more groups selected from halogen, cyano, nitro, methoxy, ethoxy, methyl, ethyl, hydroxy, and —NR⁵R⁶;

n is selected from 0, 1, 2, 3, 4, 5 and 6;

R³ is selected from hydrogen, halogen, amino, C₁₋₁₀alkyl, C₁₋₁₀alkyl-C₃₋₁₀cycloalkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₈heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from halogen, cyano, nitro, methoxy, ethoxy, methyl, ethyl, hydroxy, and —NR⁵R⁶;

wherein R⁵ and R⁶ are independently selected from —H, C₁₋₆alkyl, C₂₋₆alkenyl, C₂₋₆alkynyl, and a divalent C₁₋₆group that together with another divalent R⁵ or R⁶ may form a ring or a portion of a ring; and

R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)—; wherein said C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)—C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

The compounds of the present invention also include those of Formula II, wherein

R¹ is selected from from —H and C₁₋₆alkyl;

R² is selected from C₁₋₈alkyl, C₂₋₈alkenyl, C₂₋₈alkynyl, C₃₋₈-cycloalkyl, C₃₋₈cycloalkyl-C₁₋₄alkyl, C₃₋₆cycloalkenyl, C₃₋₆cycloalkenyl-C₁₋₄alkyl, C₃₋₆heterocyclyl, C₃₋₆heterocycloalkyl, 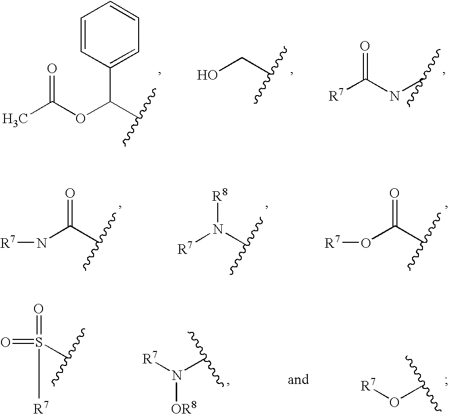
wherein said C₁₋₈alkyl, C₂₋₈alkenyl, C₂₋₈alkynyl, C₃₋₈cycloalkyl, C₃₋₈cycloalkyl-C₁₋₄alkyl, C₄₋₆cycloalkenyl, C₄₋₆cycloalkenyl-C₁₋₄alkyl, C₃₋₆heterocyclyl, C₃₋₆heterocycloalkyl 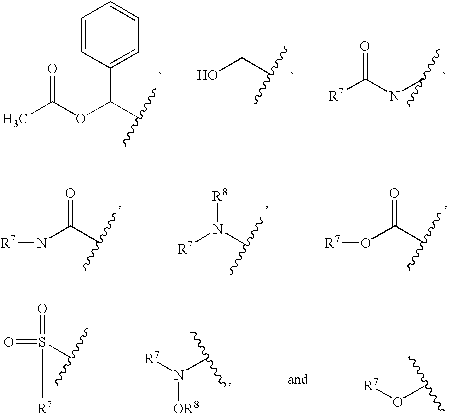
used in defining R² is optionally substituted with one or more groups selected from C₁₋₆alkyl, halogen, amino and C₁₋₆alkoxy,

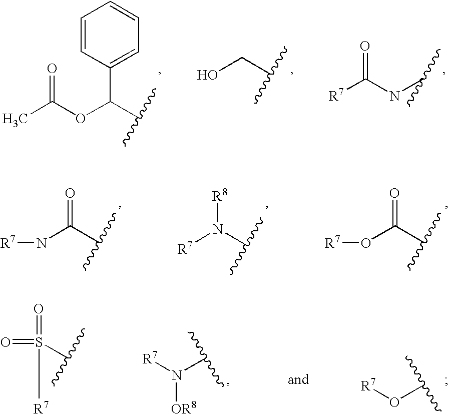

each of R⁷ and R⁸ is independently selected from —H, C₁₋₆alkyl, C₁₋₆alkoxy, C₂₋₆alkenyl, C₃₋₆cycloalkyl, C₃₋₆cycloalkyl-C₁₋₆alkyl, C₃₋₆heterocyclyl and C₃₋₆heterocylcyl-C₁₋₆alkyl, wherein said C₁₋₆alkyl, C₂₋₆alkenyl, C₃₋₆cycloalkyl, C₃₋₆cycloalkyl-C₁₋₆alkyl, C₃₋₆heterocyclyl and C₃₋₆heterocylcyl-C₁₋₆alkyl used in defining R⁷ and R⁸ are optionally substituted by one or more groups selected from halogen, cyano, nitro, methoxy, ethoxy, methyl, ethyl, hydroxy and —NR⁵R⁶;

n is selected from 0, 1, 2, 3 and 4;

R³ is selected from hydrogen, halogen, amino, C₁₋₈alkyl, C₂₋₈alkenyl, C₂₋₈alkynyl, C₃₋₈cycloalkyl, C₄₋₈cycloalkenyl, C₃₋₅heteroaryl, R⁵R⁶N—, C₃₋₆cycloalkyl-C₁₋₄alkyl, C₄₋₆cycloalkenyl-C₁₋₄alkyl, phenyl, phenyl-C₁₋₄alkyl, C₃₋₆heterocyclyl or C₃₋₆heterocyclyl-C₁₋₄alkyl; wherein said amino, C₁₋₆alkyl, C₂₋₆alkenyl, C₂₋₆alkynyl, C₃₋₆cycloalkyl, C₄₋₆cycloalkenyl, C₃₋₅heteroaryl, R⁵R⁶N—, C₃₋₆cycloalkyl-C₁₋₄alkyl, C₄₋₆cycloalkenyl-C₁₋₄alkyl, phenyl, phenyl-C₁₋₄alkyl, C₃₋₆heterocyclyl or C₃₋₆heterocyclyl-C₁₋₄alkyl used in defining R³ is optionally substituted by one or more groups selected from C₁₋₄alkyl, C₂₋₄alkenyl, halogen, C₁₋₄alkoxy, amino, nitro, cyano, oxo, methoxy, ethoxy, methyl, ethyl, hydroxy, C₁₋₆cycloalkyl-C₁₋₆alkyl, C₃₋₆heterocyclyl, C₃₋₆heterocyclyl-C₁₋₆alkyl, and —NR⁵R⁶;

wherein R⁵ and R⁶ are independently selected from from —H and C₁₋₃alkyl; and

R⁴ is selected from C₃₋₆cycloalkyl, C₃₋₆cycloalkyl-C₁₋₄alkyl, C₄₋₆cycloalkenyl, C₆₋₁₀aryl, phenyl, alkyl, phenyl-C₁₋₄alkyl, C₃₋₆heterocyclyl or C₃₋₆heterocyclyl-C₁₋₄alkyl; wherein said C₃₋₆cycloalkyl, C₃₋₆cycloalkyl-C₁₋₄alkyl, C₄₋₆cycloalkenyl, C₆₋₁₀aryl, phenyl, alkyl, phenyl-C₁₋₄alkyl, C₃₋₆heterocyclyl or C₃₋₆heterocyclyl-C₁₋₄alkyl used in defining R⁴ is optionally substituted by one or more groups selected from C₁₋₄alkyl, C₁₋₄alkoxy, halogen, cyano, amino, nitro, oxo, hydroxy, and —NR⁵R⁶.

The compounds of the present invention also include those of Formula II wherein,

R¹ is selected from —H and C₁₋₄alkyl;

R² is selected from C₁₋₆alkyl, C₂₋₆alkenyl, C₂₋₆alkynyl, C₃₋₆cycloalkyl, C₃₋₆cycloalkyl-C₁₋₄alkyl, C₃₋₆cycloalkenyl, C₃₋₆cycloalkenyl-C₁₋₄alkyl, C₃₋₆heterocyclyl, C₃₋₆heterocycloalkyl, 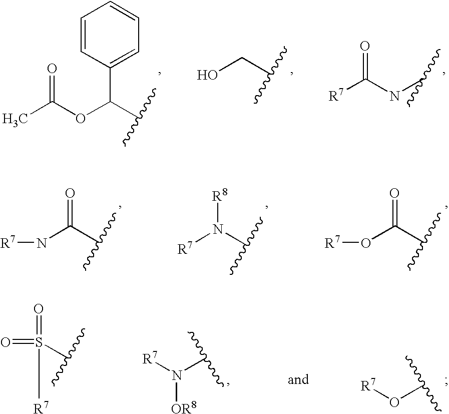
wherein said C₁₋₆alkyl, C₂₋₆alkenyl, C₂₋₆alkynyl, C₃₋₆cycloalkyl, C₃₋₆cycloalkyl-C₁₋₄alkyl, C₃₋₆cycloalkenyl, C₃₋₆cycloalkenyl-C₁₋₄alkyl, C₃₋₆heterocyclyl, C₃₋₆heterocycloalkyl, 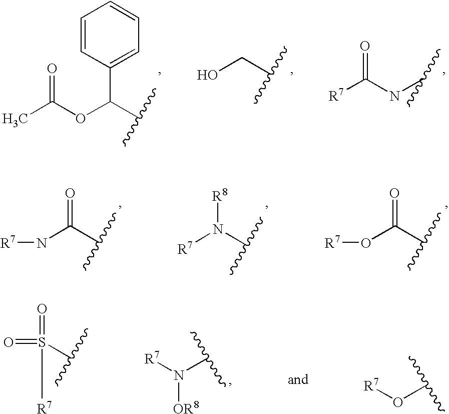
used in defining R² is optionally substituted with one or more groups selected from C₁₋₄alkyl, Cl, F, amino and C₁₋₄alkoxy,

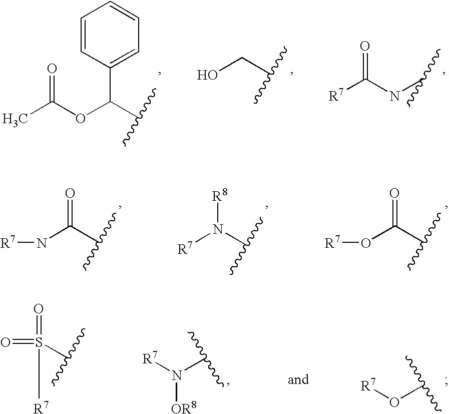

wherein each of R⁷ and R⁸ is independently selected from —H, C₁₋₆alkyl, C₂₋₆alkenyl, C₂₋₆alkynyl, C₁₋₆alkoxy and C₃₋₆cycloalkyl;

n is selected from 0, 1, 2 and 3;

R³ is selected from hydrogen, halogen, amino, C₁₋₆alkyl, C₂₋₆alkenyl, C₂₋₆alkynyl, C₃₋₆cycloalkyl, C₃₋₆cycloalkyl-C₁₋₄alkyl, C₃₋₆heterocyclyl or C₃₋₆heterocyclyl-C₁₋₄alkyl wherein said amino, C₁₋₆alkyl, C₂₋₆alkenyl, C₃₋₆cycloalkyl, C₃₋₆cycloalkyl-C₁₋₄alkyl, C₃₋₆heterocyclyl or C₃₋₆heterocyclyl-C₁₋₄alkyl used in defining R³ is optionally substituted by one or more groups selected from Cl, F, methoxy, ethoxy, methyl, ethyl and hydroxy;

R⁵ and R⁶ are independently selected from —H and C₁₋₃alkyl; and

R⁴ is selected from C₁₋₆alkyl, C₂₋₆alkenyl, C₂₋₆alkynyl, phenyl, alkyl, C₃₋₆cycloalkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, wherein said phenyl, alkyl, phenyl-C₁₋₄alkyl, C₃₋₆cycloalkyl-C₁₋₄alkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, used in defining R⁴ is optionally substituted by one or more groups selected from C₁₋₄alkyl, C₁₋₄alkoxy, halogen, amino, cyano, oxo, hydroxy, and —NR⁵R⁶.

The compounds of the present invention also include those of Formula II, wherein

R¹ is selected from —H and C₁₋₃alkyl;

R² is Cl, F, methyl, ethyl, propyl, t-butyl, n-butyl, hydroxy, methoxy, ethoxy, cyclopropyl, cyclobutyl, benzyl, phenyl, thienyl, 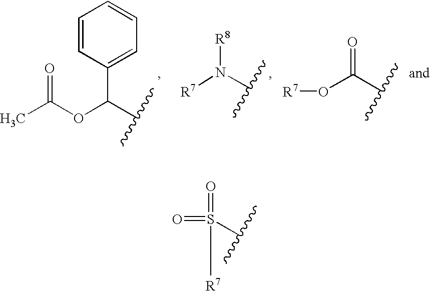
wherein said cyclopropyl, cyclobutyl, benzyl, phenyl, thienyl, 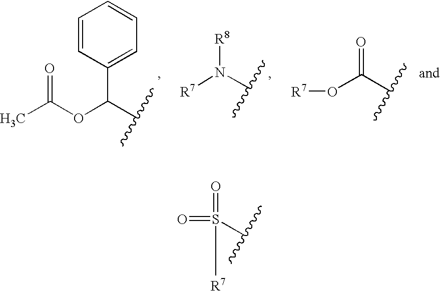
used in defining R² is optionally substituted with one or more Cl, F, C₁₋₄alkyl,

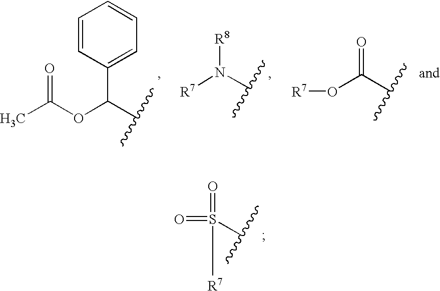

each of R⁷ and R⁸ is independently selected from —H, Cl, F, methyl, ethyl, propyl, t-butyl, n-butyl and C₁₋₄alkoxy;

n is selected from 0, 1 and 2;

R³ is selected from hydrogen and Cl; and

R⁴ is selected from C₁₋₄alkyl, C₂₋₄alkenyl, C₂₋₄alkynyl, phenyl, cyclohexyl, cyclopentyl, cyclobutyl, cyclopropyl, cyclohexyl, cyclopentyl, and benzyl; wherein said phenyl, cyclohexyl, cyclopentyl, cyclobutyl, cyclopropyl, cyclohexyl, cyclopentyl, and benzyl used in defining R⁴ is optionally substituted by one or more groups selected from fluorine, chlorine, methoxy, ethoxy, methyl, butyl, propyl, ethyl and hydroxy.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I or II. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formulae I or II. It will further be understood that the present invention encompasses tautomers of the compounds of the Formulae I or II.

It will also be understood that certain compounds of the present invention may exist in solvated, for example hydrated, as well as unsolvated forms. It will further be understood that the present invention encompasses all such solvated forms of the compounds of the Formulae I or II.

Within the scope of the invention are also salts of the compounds of the Formulae I or II. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formulae I or II above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as modulators or ligands such as agonists, partial agonists, inverse agonist or antagonists of CB1 receptors. More particularly, the compounds of the invention exhibit selective activity as agonist of the CB1 receptors and are useful in therapy, especially for relief of various pain conditions such as chronic pain, neuropathic pain, acute pain, cancer pain, pain caused by rheumatoid arthritis, migraine, visceral pain etc. This list should however not be interpreted as exhaustive. Additionally, compounds of the present invention are useful in other disease states in which dysfunction of the CB1 receptor is present or implicated. Furthermore, the compounds of the invention may be used to treat cancer, multiple sclerosis, Parkinson's disease, Huntington's chorea, Alzheimer's disease, anxiety disorders, gastrointestinal disorders and cardiavascular disorders.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of opioid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhoea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastrointestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to Formulae I or II above, for the manufacture of a medicament for the treatment of any of the conditions discussed above.

A further aspect of the invention is a method for the treatment of a subject suffering from any of the conditions discussed above, whereby an effective amount of a compound according to Formulae I or II above, is administered to a patient in need of such treatment.

Thus, the invention provides a compound of Formulae I or II, or pharmaceutically acceptable salt or solvate thereof, as hereinbefore defined for use in therapy.

In a further aspect, the present invention provides the use of a compound of Formulae I or II, or a pharmaceutically acceptable salt or solvate thereof, as hereinbefore defined in the manufacture of a medicament for use in therapy.

In the context of the present specification, the term “therapt” also includes “prophylaxis” unless there are specific indications to the contrary. The term “therapeutic” and “therapeutically” should be continued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, acute pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administrator may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (per cent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formulae I or II as defined above for the manufacture of a medicament.

Also within the scope of the invention is the use of any compound of Formulae I or II for the manufacture of a medicament for the therapy of pain.

Additionally provided is the use of any compound according to Formulae I or II for the manufacture of a medicament for the therapy of various pain conditions including, but not limited to: acute pain, chronic pain, neuropathic pain, acute pain, back pain, cancer pain, and visceral pain.

A further aspect of the invention is a method for therapy of a subject suffering from any of the conditions discussed above, whereby an effective amount of a compound according to Formulae I or II above, is administered to a patient in need of such therapy.

Additionally, there is provided a pharmaceutical composition comprising a compound of Formulae I or II, or a pharmaceutically acceptable salt thereof, in association with a pharmaceutically acceptable carrier.

Particularly, there is provided a pharmaceutical composition comprising a compound of Formulae I or II, or a pharmaceutically acceptable salt thereof, in association with a pharmaceutically acceptable carrier for therapy, more particularly for therapy of pain.

Further, there is provided a pharmaceutical composition comprising a compound of Formulae I or II, or a pharmaceutically acceptable salt thereof, in association with a pharmaceutically acceptable carrier use in any of the conditions discussed above.

In a further aspect, the present invention provides a method of preparing the compounds of the present invention.

In one embodiment, the invention provides a process for preparing a compound of Formula IA, comprising of the step of 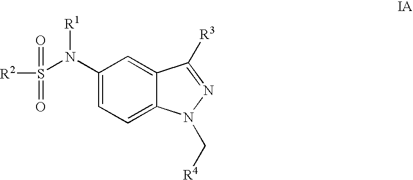
reacting a compound of Formula IB, 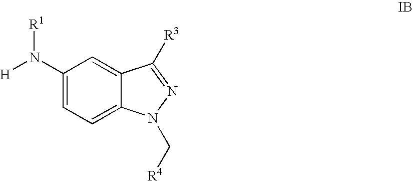
with a compound of R²SO₂Cl in the presence of triethylamine and dichloroethane, wherein

R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;

R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl used in defining R² is optionally substituted with one or more groups selected from C₁₋₆alkyl, halogen, amino and C₁₋₆alkoxy,

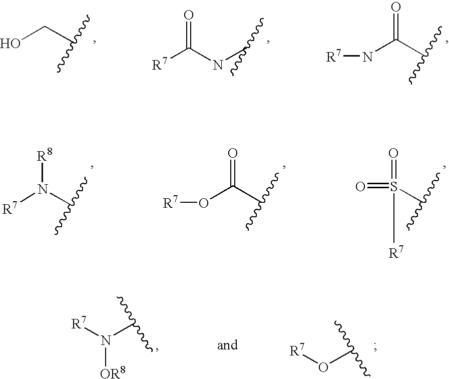

each of R⁷ and R⁸ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆group that together with another divalent group selected from R⁷ and R⁸ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and divalent C₁₋₆group used in defining R⁷ and R⁸ is optionally substituted by one or more groups selected from halogen, cyano, nitro, methoxy, ethoxy, methyl, ethyl, hydroxy, and —NR⁵R⁶;

R³ is selected from hydrogen, halogen, amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from C₁₋₄alkyl, C₂₋₄alkenyl, C₂₋₄alkynyl, halogen, C₁₋₄alkoxy, amino, nitro, cyano, oxo, hydroxy, C₁₋₆cycloalkyl-C₁₋₆alkyl, C₃₋₆heterocyclyl, C₃₋₆heterocyclyl-C₁₋₆alkyl, and —NR⁵R⁶

wherein R⁵ and R⁶ are independently selected from —H, C₁₋₈alkyl, C₂₋₈alkenyl, C₂₋₈alkynyl, and a divalent C₁₋₈group that together with another divalent R₈ or R⁶ may form a ring or a portion of a ring; and

R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)—; wherein said C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

In another embodiment, the invention provides a process for preparing a compound of Formula IIA, comprising of the step of 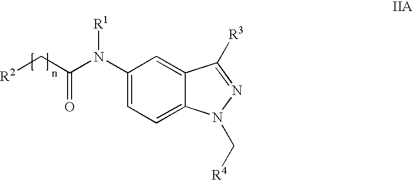
reacting a compound of Formula IIB, 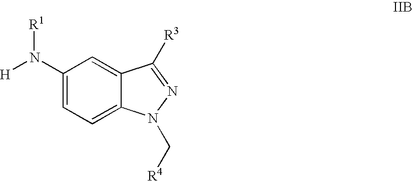
with a compound of R²COCl or PR²NCO in the presence of triethylamine and dichloroethane, wherein

R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;

R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl, 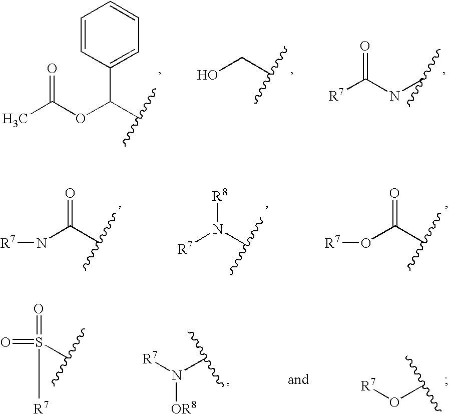
wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl 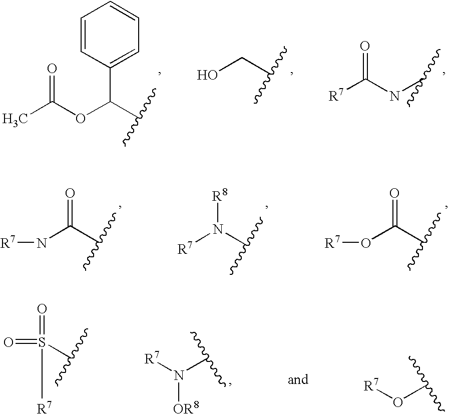
used in defining R² is optionally substituted with one or more groups selected from C₁₋₆alkyl, halogen, amino and C₁₋₆alkoxyl,

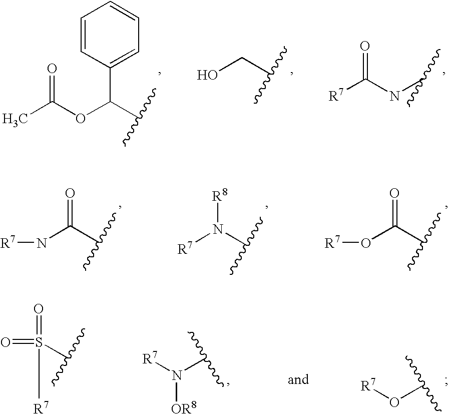

each of R⁷ and R⁸ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆group that together with another divalent group selected from R⁸ and R⁹ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and divalent C₁₋₆group is optionally substituted by one or more groups selected from halogen, cyano, nitro, methoxy, ethoxy, methyl, ethyl, hydroxy, and —NR⁵R⁶;

n is selected from 0, 1, 2, 3, 4, 5 and 6;

R³ is selected from hydrogen, halogen, amino, C₁₋₁₀alkyl, C₁₋₁₀alkyl-C₃₋₁₀cycloalkyl, C₂₋₁₀alkenyl, C₂₋₁₀alkynyl, C₃₋₁₀cycloalkyl, C₃₋₁₀cycloalkyl-C₁₋₆₋alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from halogen, cyano, nitro, methoxy, ethoxy, methyl, ethyl, hydroxy, and —NR⁵R⁶;

wherein R⁵ and R⁶ are independently selected from —H, C₁₋₆alkyl, C₂₋₆alkenyl, C₂₋₆alkynyl, and a divalent C₁₋₆group that together with another divalent R⁵ or R⁶ may form a ring or a portion of a ring; and

R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)—C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)—; wherein said C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

Further embodiments of the invention provide a process for preparing the compounds of the invention according to the synthetic routes depicted in the general procedures below:

Compounds of the present invention may be prepared according to the synthetic routes as depicted in the following schemes. 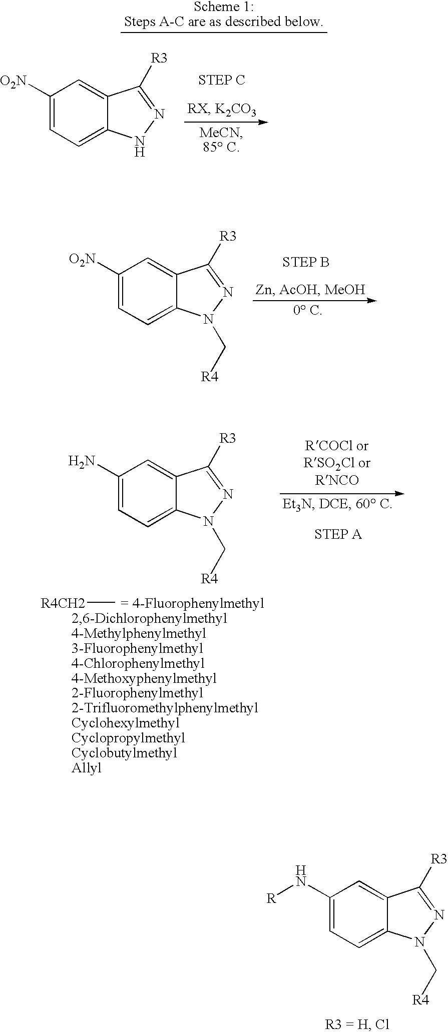
Procedure—Step A (Aminoindazole Reaction with Electrophile):
The acetic acid salt of the aminoindazole was dissolved in dichloromethane (30 ml/mmol aminoindazole). A 1N aqueous NaOH solution (10 ml/mmol aminoindazole) was added and the mixture was stirred at room temperature for 15 minutes. The mixture was filtered on Hydromatrix. The filtrate was concentrated in vacuo to yield the free base of the aminoindazole.
To a solution of the free aminoindazole (1 equiv.) and triethylamine (2 equiv.) in dichloroethane (15 ml/mmol aminoindazole) was added the electrophile (1.1 equiv.). The reaction mixture was heated at 60° C. until all the aminoindazole had been consumed (typically overnight). The reaction was cooled to room temperature. The reaction mixture was diluted with dichloromethane. The organic phase was washed with a 1N aqueous NaOH solution. The mixture was filtered on Hydromatrix and concentrated in vacuo. The residue was purified by reverse phase HPLC (gradient 30-80% CH₃CN in H₂O) to provide the TFA salt of the title compound after lyophilysation.
Procedure Step B: (Nitro Reduction):
To a suspension of nitroindazole (1 equiv.) in glacial acetic acid (6 ml/mmol nitroindazole) and methanol (6 ml/mmol indazole) at 0° C. was added zinc powder (5 equiv.). The reaction mixture was stirred at 0° C. until all of the nitroindazole had been consumed (typically 30 minutes). Methanol was added to the reaction. The reaction mixture was filtered on a celite pad, which was washed several times with methanol. The filtrate was concentrated in vacuo. Dichloromethane was added to the residue and the mixture was filtered to remove Zn(OAc)₂. The filtrate was concentrated in vacuo.
Procedure Step C: (Allylation):
To a suspension of indazole (1 equiv.) and potassium carbonate (2 equiv.) in acetonitrile (7 ml/mmol indazole) was added the halide (1.1 equiv.). The reaction mixture was heated at reflux until all of the indazole had been consumed (typically between 2 h and 6 h). The reaction was cooled to room temperature. Dichloromethane (7 ml/mmol indazole) was added. The reaction was stirred at room temperature for 15 minutes to dissolve the product. The product was next filtered, and then concentrated in vacuo.
Biological Evaluation
hCB₁ and hCB₂ Receptor Binding

Human CB1 receptor from Receptor Biology (hCB1) or human CB₂ receptor from BioSignal (hCB2) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC₅₀ of the compounds of the invention at hCB₁ and hCB₂ are evaluated from 10-point dose-response curves done with ³H-CP55,940 at 20000 to 25000 dpm per well (0.17-0.21 nM) in a final volume of 300 l. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the dTomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

hCB₁ and hCB₂ GTPγS Binding

Human CB₁ receptor from Receptor Biology (hCB1) or human CB₂ receptor membranes (BioSignal) are thawed at 37° C., passed 3 times through a 25-gauge blunt-end needle and diluted in the GTPγS binding buffer (50 mM Hepes, 20 mM NaOH, 100 mM NaCl, 1 mM EDTA, 5 mM MgCl₂, pH 7.4, 0.1% BSA). The EC₅₀ and Eₘₐₓ of the compounds of the invention are evaluated from 10-point dose-response curves done in 300 μl with the appropriate amount of membrane protein and 100000-130000 dpm of GTPg³⁵S per well (0.11-0.14 nM). The basal and maximal stimulated binding is determined in absence and presence of 1 μM (hCB₂) or 10 μM (hCB₁) Win 55,212-2 respectively. The membranes are pre-incubated for 5 minutes with 56.25 μM (hCB2) or 112.5 μM (hCB₁) GDP prior to distribution in plates (15 μM (hCB₂) or 30 μM (hCB₁) GDP final). The plates are vortexed and incubated for 60 minutes at room temperature, filtered on Unifilters GF/B (presoaked in water) with the Tomtec or Packard harvester using 3 ml of wash buffer (50 mM Tris, 5 mM MgCl₂, 50 mM NaCl, pH 7.0). The filters are dried for 1 hour at 55° C. The radioactivity (cpm) is counted in a TopCount (Packard) after adding 65 μl/well of MS-20 scintillation liquid. Antagonist reversal studies are done in the same way except that (a) an agonist dose-response curve is done in the presence of a constant concentration of antagonist, or (b) an antagonist dose-response curve is done in the presence of a constant concentration of agonist.

Based on the above assays, the dissociation constant (Ki) for a particular compound of the invention towards a particular receptor is determined using the following equation:
Ki=IC₅₀/(1+[rad]/Kd),

Wherein IC₅₀ is the concentration of the compound of the invention at which 50% displacement has been observed;

[rad] is a standard or reference radioactive ligand concentration at that moment; and

Kd is the dissociation constant of the radioactive ligand towards the particular receptor.

Using above-mentioned assays, the Ki towards human CB₁ receptors for most compounds of the invention is measured to be in the range of 36-5700 nM. The Ki towards human CB₂ receptors for most compounds of the invention is measured to be in the range of about 1.6-36 nM.

### EXAMPLES

The invention will further be described in more detail by the following Examples which describe methods whereby compounds of the present invention may be prepared, purified, analyzed and biologically tested, and which are not to be construed as limiting the invention.

Where retention time (t_{R}) values are provided the LC/MS conditions were as follows: column: Phenomenex Synergy 4u Polar-RP 80A, 30×2.00mm; mobile phase A: 0.1% TFA in water, B: 0.1% TFA in acetonitrile; gradient: 90:10 (A/B) linear to 5:95 (A/B) in 2.25 min., hold for 0.75 min., re-equilibration time: 0.5 min. at 10% B. Where capacity factor (k′) provided, the LC/MS conditions were as follows: column: Zorbax C-18; mobile phase: A—0.05% TFA in water, B—0.05% TFA in acetonitrile; gradient: 10-95% B, 1 mL/min, 40° C.

### Example 1

### N-[3-chloro-1-(3-fluorobenzyl)-1H-indazol-5-yl]-2-methoxyacetamide

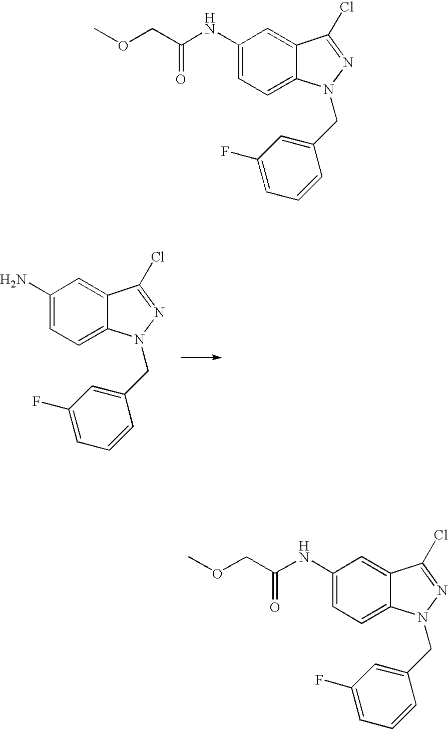 Following General Procedure Step A, The title compound (10 mg, 24%) was obtained as a white solid. ¹H-NMR (CDCl₃): δ ppm 3.53 (s, 3 H) 4.06 (s, 2 H) 5.50 (s, 2 H) 6.88 (d, J=9.37 Hz, 1 H) 6.95-7.00 (m, 2 H) 7.25-7.31 (m, 2 H) 7.55 (dd, J=9.08, 1.86 Hz, 1 H) 7.97 (d, J=1.56 Hz, 1 H) 8.36 (s, 1 H). MS (ESI) (M+H)⁺=348; k′: 5.07.
Step B:

### Example

### 3-chloro-1-(3-fluorobenzyl)-5-amino-1H-indazole

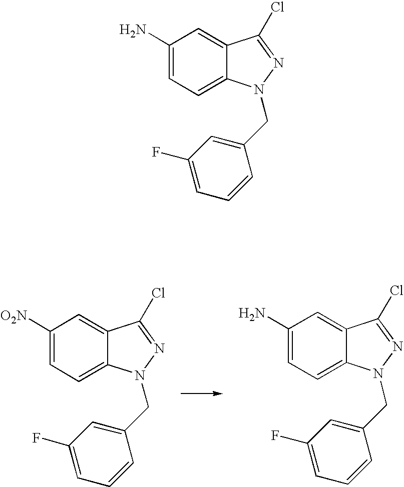 Following General Procedure Step B, the acetic acid salt of the title compound (3.06 g, 50%) was obtained as a white solid. ¹H-NMR (CDCl₃): δ ppm 1.57 (s, 6 H) 5.45 (s, 2 H), 6.85-6.87 (m,3 H), 6.93-6.98 (m, 2 H), 7.09-7.11 (m, 1 H), 7.24-7.30 (m, 1 H). MS (ESI) (M+H)⁺=276.
Step C:

### Example

### 3-chloro-1-(3-fluorobenzyl)-5-nitro-1H-indazole

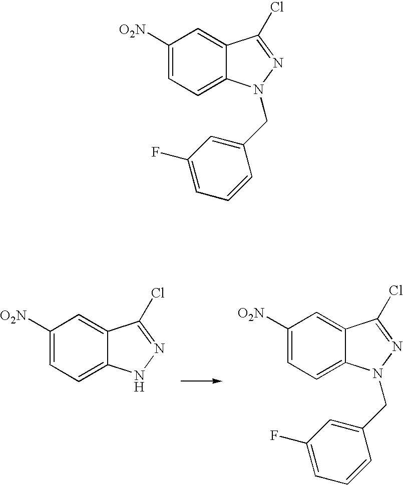 The crude compound obtained following the General Procedure Step C was triturated in methanol and filtered. The filtrate was concentrated in vacuo and the procedure was repeated. The title compound (4.77 g, 77%) was obtained as a yellow solid.

### Example 2

### N-[3-chloro-1-(4-methylbenzyl)-1H-indazol-5-yl]-2-methoxyacetamide

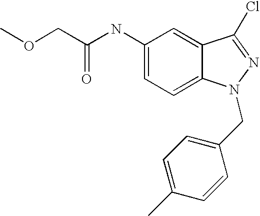 Following General Procedure Step A, the title compound (20 mg, 48%) was obtained as a white solid. ¹H-NMR (CDCl₃): δ ppm 2.31 (s, 3 H), 3.52 (s, 3 H), 4.05 (s, 2 H), 5.47 (s, 2 H), 7.11 (s, 4 H), 7.24-7.27 (m, 1 H), 7.51 (dd, J=8.98 Hz, 1.95 Hz, 1 H), 7.93 (d, J=1.56 Hz, 1 H), 8.33 (s, 1 H). MS (ESI) (M+H)⁺=344. Anal. Calcd for C₁₈H₁₈ClN₃O₂+0.1 TFA: C, 61.54; H 5.14; N 11.83. Found: C, 61.76; H 5.30; N 11.85.
Step B:

### Example

### 3-chloro-1-(4-methylbenzyl)-5-amino-1H-indazole

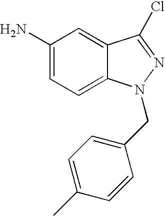

The crude compound obtained following General Procedure Step B was triturated in diethyl ether and filtered. The filtrate was concentrated in vacuo and the procedure was repeated twice. The acetic acid salt of the title compound (3.38 g, 62%) was obtained as a white solid.

¹H-NMR (CD₆OD): δ ppm 1.98 (s, 6 H), 2.28 (s, 3 H), 5.43 (s, 2 H), 6.84 (d, J=1.56 Hz, 1 H), 6.97 (dd, J=8.98 Hz, 1.95 Hz, 1 H), 7.06-7.12 (m, 4 H), 7.29 (d, J=8.98 Hz, 1 H). MS (ESI) (M+H)⁺=272.

Step C:

### Example

### 3-chloro-1-(4-methylbenzyl)-5-nitro-1H-indazole

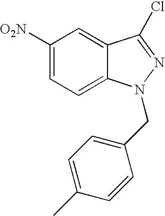 The crude compound obtained following General Procedure Step C was triturated in methanol and filtered. The filtrate was concentrated in vacuo and the procedure was repeated. The title compound (4.22 g, 69%) was obtained as a yellow solid.

### Example 3

### N-[1-alkyl-3-chloro-1H-indazol-5-yl]-2-methoxyacetamide

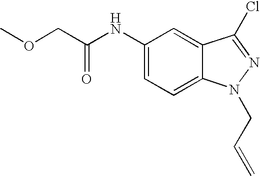 Following General Procedure step A, the title compound (20 mg, 35%) was obtained as a white solid. ¹H-NMR (CDCl₃): δ ppm 3.53 (s, 3 H), 4.06 (s, 2 H), 4.94 (d, J=5.66 Hz, 2 H), 5.18 (d, J=17.18 Hz, 1 H), 5.25 (d, J=9.57 Hz, 1 H), 5.95-6.05 (m, 1 H), 7.34 (d, J=8.98 Hz, 1 H), 7.57 (dd, J=8.98 Hz, 1.56 Hz, 1 H), 7.95 (s, 1 H), 8.36 (s, 1 H). MS (ESI) (M+H)⁺=280. Anal. Calcd for C₁₃H₁₄ClN₃O₂+0.1 TFA: C, 54.46; H 4.88; N 14.43. Found: C, 54.78; H 5.12; N 14.48.
Step B:

### Example

### 1-alkyl-3-chloro-5-amino-1H-indazole

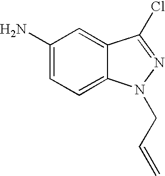 The crude compound obtained following the general procedure step B was dissolved in dichloromethane. The organic phase was washed with a 1N aqueous NaOH solution and brine. The organic phase was dried over Na₂SO₄, filtered, and concentrated in vacuo. The crude compound was purified by flash chromatography (1:99 methanol:dichloromethane to 3:97 methanol:dichloromethane). The title compound (2.65 g, 79%) was obtained as a pink solid. ¹H-NMR (CD₃OD): δ ppm 4.90 (m, 2 H), 5.06 (dd, J=17.18 Hz, 1.17 Hz, 2 H), 5.18 (dd, J=10.15 Hz, 0.98 Hz, 2 H), 5.98 (ddd, J=22.36 Hz, 10.45 Hz, 5.47 Hz, 1 H), 6.84 (d, J=1.95 Hz, 1 H), 7.01 (dd, J=8.88 Hz, 2.05 Hz, 1 H), 7.32 (d, J=8.98 Hz, 1 H). MS (ESI) (M+H)⁺=208.
Step C:

### Example

### 1-alkyl-3-chloro-5-nitro-1H-indazole

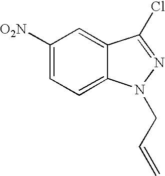 The crude compound obtained following the General Procedure Step C was purified by flash chromatography (dichloromethane). The title compound (3.95 g, 82%) was obtained as a yellow solid.

### Example 4

### N-[1-cyclohexylmethyl-6-methoxy-1H-indazol-5-yl]benzenesulfonamide

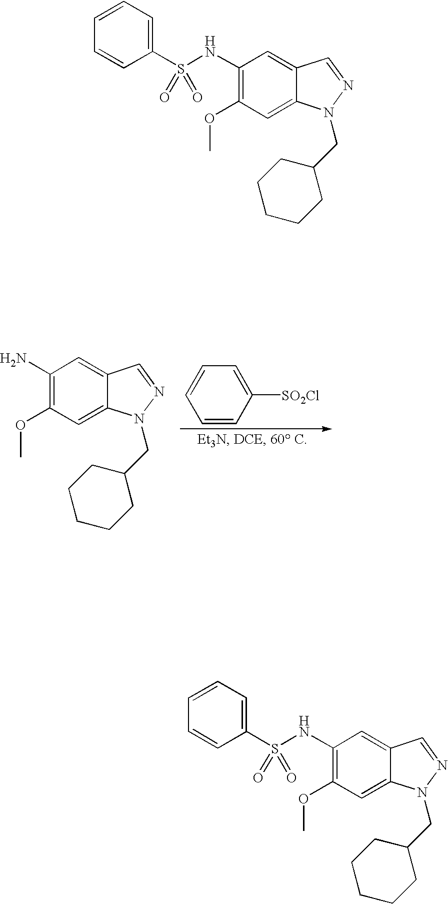 Step A:
To a solution of the free aminoindazole (72 mg, 278 μmol) and triethylamine (116 μl, 834 μmol) in dichloroethane (5 ml) was added phenylsulfonyl chloride (42 μl, 334 μmol). The reaction mixture was heated at 60° C. for 2 h. The reaction was cooled to room temperature. The reaction mixture was diluted with dichloromethane. The organic phase was washed with a 1N aqueous NaOH solution. The aqueous layer was extracted with more dichloromethane. The organic phases were combined, dried over Na₂SO₄, filtered, and concentrated in vacuo. The residue was purified by reverse phase HPLC (gradient 40-95% CH₃CN in H₂O), concentrated in vacuo and lyophilysed. The residue was dissolved in a minimum amount of dichloromethane. A 1N HCl solution in diethylether (1 ml) was added and the solvent was evaporated. The HCl salt of the title compound (90 mg, 74%) was obtained as a white solid.

¹H-NMR (CDCl₃): δ ppm 1.01-1.11 (m, 2 H), 1.15-1.23 (m, 3 H), 1.53-1.57 (m, 2 H), 1.62-1.72 (m, 3 H), 1.92-2.02 (m, 1 H), 3.78 (s, 3 H), 4.20 (d, J=7.23 Hz, 2 H), 7.21 (d, J=8.79 Hz, 1 H), 7.46 (t, J=7.62 Hz, 2 H), 7.48 (d, J=8.79 Hz, 1 H), 7.56 (t, J=7.32 Hz, 1 H), 7.68 (d, J=7.23 Hz, 2 H), 8.10 (s, 1 H). Anal. Calcd for C₁₃H₁₄ClN₃O₂+0.1 TFA: C, 54.46; H 4.88; N 14.43. Found: C, 54.78; H 5.12; N 14.48.

Step B:

### Example

### 1-(cyclohexylmethyl)-6-methoxy-1H-indazole-5-amine

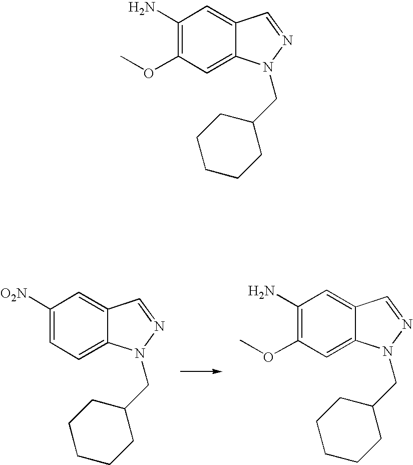 The crude compound obtained as a by-product of general procedure step B. It was purified by flash chromatography (1:1:98 methanol:triethylamine:dichloromethane). ¹H-NMR (Acetone-d₆): δ ppm 0.96-1.05.(m, 2 H), 1.12-1.26 (m, 3 H), 1.57-1.71 (m, 5 H), 1.91-2.03 (m, 1 H), 3.09 (s, 2 H), 4.11 (d, J=7.22 Hz, 2 H), 4.13 (s, 3 H), 6.92 (s, 2 H), 7.98 (s, 1 H). MS (ESI) (M+H)⁺=260.
Step C:

### Example

### 1-(cyclohexylmethyl)-5-nitro-1H-indazole

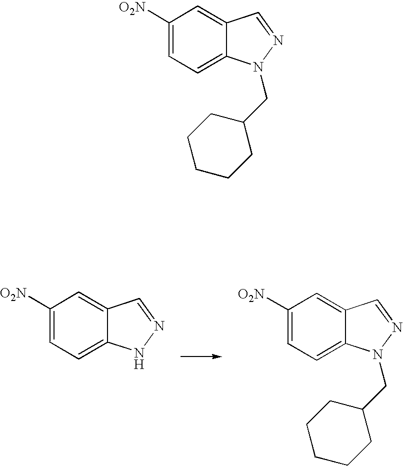 The crude compound obtained following the General Procedure step C was purified by flash chromatography (1:4 EtOAc:hexane) to separate the isomers (desired isomer is less polar). The title compound (8.54 g, 54%) was obtained as a pale yellow solid.

### Example 5

### N-{1-[(6-chloropyridin-3-yl)methyl]-1H-indazol-5-yl}benzenesulfonamide

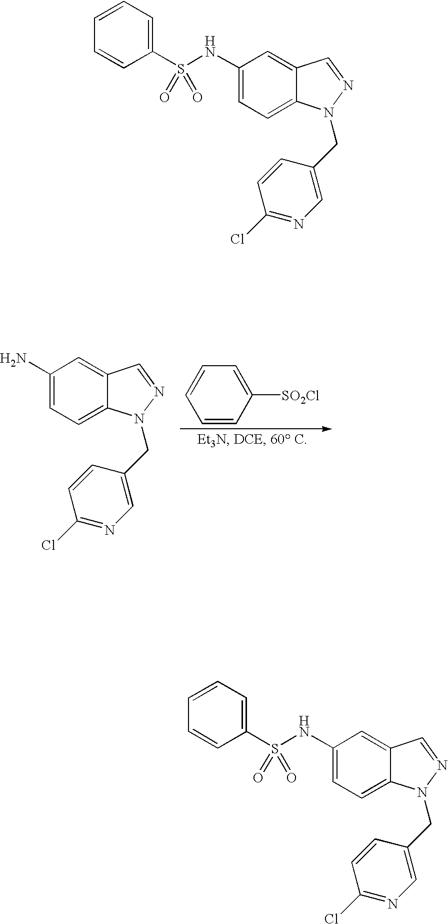 Step A:

To a solution of the aminoindazole (292 mg, 861 μmol) in dichloroethane (9 ml) was added phenylsulfonyl chloride (132 μl, 1.03 mmol). The reaction mixture was heated at 60° C. overnight. The reaction was cooled to room temperature. The reaction mixture was diluted with dichloromethane. The organic phase was washed with a sat. NaHCO₃. The organic phase was dried over Na₂SO₄, filtered, and concentrated in vacuo. The residue was purified by flash chromatography (1:99 to 3:97 methanol: dichloromethane). The desired compound was further purified by crystallisation in dichloromethane. The title compound (236 mg, 69%) was obtained as a white solid. ¹H-NMR (Acetone d6): δ ppm 5.67 (s, 2 H), 7.23 (dd, J=8.88, Hz 1.86 Hz, 1 H) , 7.37 (d, J=8.20 Hz, 1 H), 7.46 (t, J=7.62 Hz, 2 H), 7.53-7.56 (m, 2 H), 7.58 (d, J=8.79 Hz, 1 H), 7.65 (dd, J=8.30 Hz, 2.44 Hz, 1 H), 7.71 (d, J=8.01 Hz, 2 H), 7.99 (s, 1 H), 8.36 (d, J=1.95 Hz, 1 H), 8.91 (s, 1 H). MS (ESI) (M+H)⁺=399.1. Anal. Calcd for C₁₉H₁₅N₄O₂SCl+0.1 DCM: C, 56.44; H 3.75; N 13.71. Found: C, 56.59; H 3.81; N 14.05.

Step B:

### Example

### 1-[(6-chloropyridin-3-yl)methyl]-1H-indazol-5-amine

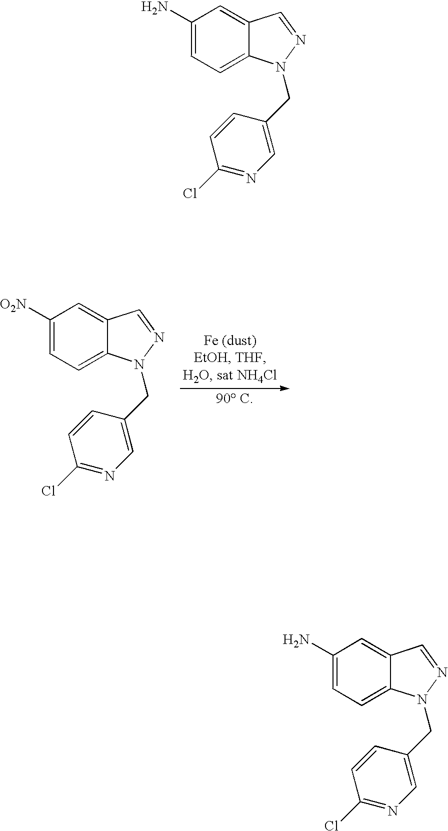 To a solution of nitroindazole (5.82 g, 20.2 mmol) in a mixture of ethanol: THF: H₂O: sat NH₄Cl (200 ml, 4:2:1:1) was added iron dust (2.5 g, 44.4 mmol). The reaction mixture was stirred at 90° C. for 5 hours. The reaction was cooled to room temperature and methanol was added to the mixture. The reaction mixture was filtered on a celite pad, which was washed many times with methanol. The filtrate was concentrated in vacuo. The residue was dissolved in dichloromethane and NaOH 1N, extracted with dichloromethane. The organic phases were combined, dried over Na₂SO₄, filtered, and concentrated in vacuo. The crude compound (4.25 g, 82%) was used directly for the next step.
Step C:

### Example

### 1-[(6-chloropyridin-3-yl)methyl]-5-nitro-1H-indazole

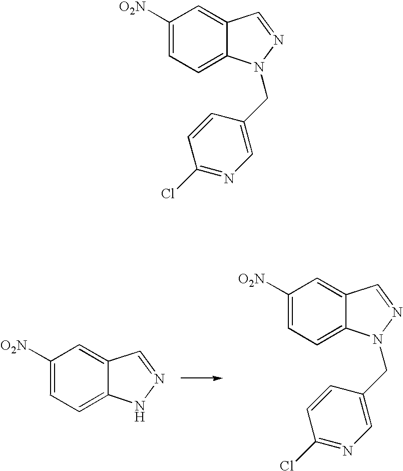 The crude compound obtained following General Procedure step C was purified by flash chromatography (1:1 ethyl acetate:hexane to ethyl acetate) to separate the isomers (desired isomer is less polar). MS (ESI) (M+H)⁺=289.
Procedure for Synthesis of Acylated Indazoles in a Microtiter Plate—Examples 6-19 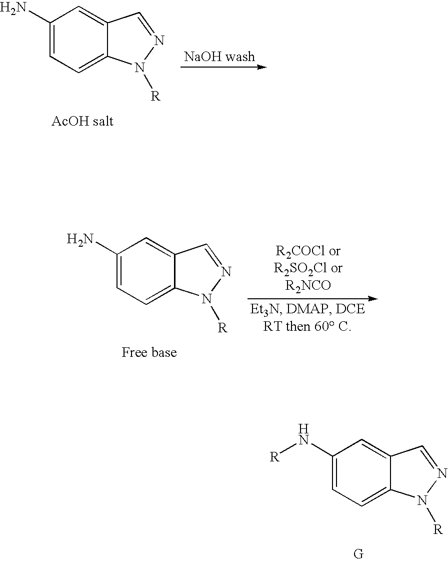
General Procedure for Step A in Microtiter Plate:

The acetic acid salt of the aminoindazole was dissolved in dichloromethane (20 ml/mmol aminoindazole). The organic phase was washed with a 1N aqueous NaOH solution (10 ml/mmol aminoindazole), dried over Na₂SO₄, filtered, and concentrated in vacuo to yield the free base of the aminoindazole.

To a solution of the free aminoindazole (150 μmoles in 800 μl DCE) was added triethylamine (63 μl, 3 equiv.), followed by a solution of the acylating reagent (isocyanate, sulfonyl chloride or acyl chloride) (1.1 equiv., 165 μmol in 660 μl DCE) and a solution of DMAP (0.1 equiv., 15 μmol in 30 μl DCE). The plates were stirred at 60° C. overnight. The reaction was cooled to room temperature. The solvent was evaporated in vacuo. The residues were dissolved in 1.2 ml dichloromethane, washed with 200 μl NaOH (1N). The phases were separated and the aqueous phase was extracted again with 2×200 μl dichloromethane. The organics were combined and evaporated in vacuo. The residues were purified by reverse phase HPLC (gradient 40-90% CH₃CN in H₂O) to provide the TFA salts of the title compounds.

### Example 6

### 2,6-dichloro-N-[3-chloro-1-(cyclopropylmethyl)-1H-indazol-5-yl]benzamide

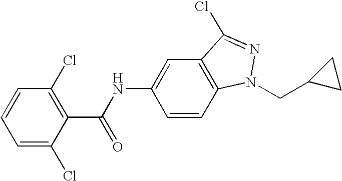 Step A:
Following the General Procedure for Step A in microtiter plate obtained the desired product.
MS (ESI) (M+H)⁺=393.7.
Step B:

### Example

### 3-chloro-1-(cyclopropylmethyl)-5-amino-1H-indazole

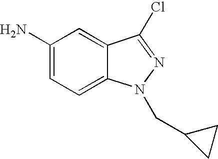 The crude compound obtained following the General Procedure step B was triturated in diethyl ether and filtered. The acetic acid salt of the title compound (1.00 g, 23%) was obtained as a pink solid. ¹H-NMR (CD₃OD): δ ppm 0.39 (dt, J=6.00, 4.61 Hz, 2 H) 0.54 (ddd, J=8.06, 6.20, 4.49 Hz, 2 H) 1.24-1.30 (m, 1 H) 1.98 (s, 6 H) 4.15 (d, J=6.83 Hz, 2 H) 6.85 (dd, J=2.05, 0.68 Hz, 1 H) 7.02 (dd, J=8.98, 2.15 Hz, 1 H) 7.38 (dd, J=8.98, 0.78 Hz, 1 H). MS (ESI) (M+H)⁺=222.
Step C:

### Example

### 3-chloro-1-(cyclopropylmethyl)-5-nitro-1H-indazole

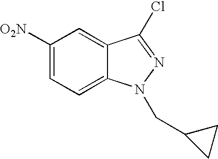 The crude compound obtained following the General Procedure step C was purified by flash chromatography (1:2 dichloromethane:toluene) to separate the isomers (desired isomer is less polar). The title compound (3.16 g, 62%) was obtained as a yellow solid. The structure of both isomers was confirmed by NOE. ¹H-NMR (DMSO-d₆): δ ppm 0.43 (d, J=4.39 Hz, 2 H), 0.52 (d, J=7.02 Hz, 2 H), 1.27-1.32 (m, 1 H), 4.37 (d, J=7.89 Hz, 2 H), 8.02 (d, J=9.65 Hz, 1 H), 8.29 (d, J=8.77 Hz, 1 H), 8.58 (s, 1 H). MS (ESI) (M+H)⁺=252.

### Example 7

### N-[1-(4-fluorobenzyl)-1H-indazol-5-yl]-2-(2-thienyl)acetamide

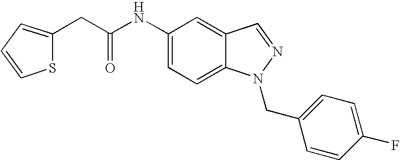 Step A:
Following Example 6 step A obtained the desired product. MS (ESI) (M+H)⁺=366.1.
Step B:

### Example

### 1-(4-fluorobenzyl)-5-amino-1H-indazole

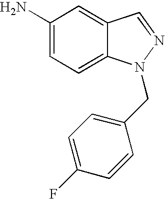 The crude compound obtained following the general procedure step B was triturated in diethyl ether and filtered. The acetic acid salt of the title compound (7.69 g, 82%) was obtained as a pink solid. ¹H-NMR (CD₃OD): δ ppm 1.98 (s, 6 H) 5.53 (s, 2 H) 6.95-7.02 (m, 4 H) 7.18 (dd, J=8.40 Hz, 5.47 Hz, 2 H) 7.32 (d, J=8.79 Hz, 1 H) 7.81 (s, 1 H). MS (ESI) (M+H)⁺=242.
Step C:

### Example

### 1-(4-fluorobenzyl)-5-nitro-1H-indazole

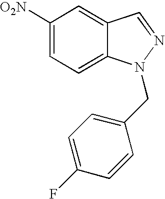 The crude compound obtained following the general procedure step C was purified by flash chromatography (1:99 methanol:dichloromethane) to separate the isomers (desired isomer is less polar). The title compound (7.01 g, 60%) was obtained as a white solid. The structure of both isomers was confirmed by NOE. ¹H-NMR (DMSO-d₆): δ ppm 5.75 (s, 2 H), 7.15 (t, J=8.33 Hz, 2 H), 7.32 (m, 2 H), 7.98 (d, J=8.77 Hz, 1 H), 8.24 (d, J=8.77 Hz, 1 H), 8.45 (s, 1 H), 8.84 (s, 1 H). MS (ESI) (M+H)⁺=272.

### Example 8

### N-(tert-butyl)-N′-[3-chloro-1-(cyclopropylmethyl)-1H-indazol-5-yl]urea

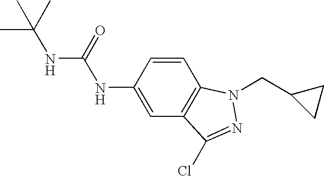 Step A: Following Example 6 step A obtained the desired product. MS (ESI) (M+H)⁺=320.9.
Step B: Example 6 step B
Step C: Example 6 step C

### Example 9

### Methyl 3-({[3-chloro-1-(cyclopropylmethyl)-1H-indazol-5-yl]amino}sulfonyl)thiophene-2-carboxylate

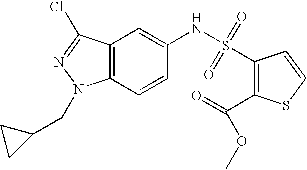 Step A. Following Example 6 step A obtained the desired product. MS (ESI) (M+H)⁺=425.7; t_{R} (min): 1.78
Step B: Example 6 step B
Step C: Example 6 step C

### Example 10

### N-[3-chloro-1-(cyclopropylmethyl)-1H-indazol-5-yl]-2-(methylsulfonyl)benzenesulfonamide

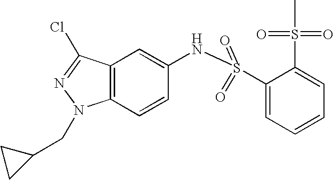 Step A: Following Example 6 step A obtained the desired product. MS (ESI) (M+H)⁺=439.7.
Step B: Example 6 step B
Step C: Example 6 step C

### Example 11

### N-[3-chloro-1-(cyclopropylmethyl)-1H-indazol-5-yl]propanamide

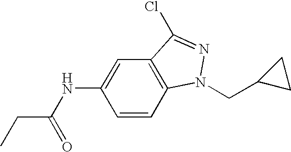 Step A: Following Example 6 step A obtained the desired product. MS (ESI) (M+H)⁺=277.9.
Step B: Example 6 step B
Step C: Example 6 step C

### Example 12

### N-(tert-butyl)-N′-[3-chloro-1-(cyclobutylmethyl)-1H-indazol-5-yl]urea

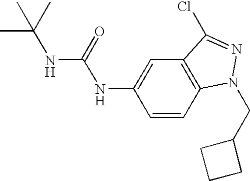 Step A: Following Example 6 step A obtained the desired product. MS (ESI) (M+H)⁺=335.6.
Step B: Example: 3-chloro-1-(cyclobutylmethyl)-5-amino-1H-indazole 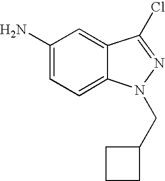
The crude compound obtained following the general procedure step B was triturated in diethyl ether and filtered. The acetic acid salt of the title compound (1.45 g, 38%) was obtained as a pink solid. ¹H-NMR (CDCl₃): δ ppm 1.76-1.92 (m, 4 H), 1.99-2.05 (m, 2 H), 2.10 (s, 3 H), 2.87 (ddd, J=22.46 Hz, 14.94 Hz, 7.52 Hz, 1H), 4.25 (d, J=7.22 Hz, 2 H), 6.84 (d, J=1.76 Hz, 1H), 6.89 (dd, J=8.89 Hz, 2.05 Hz, 1H), 7.22 (d, J=8.79 Hz, 1H). MS (ESI) (M+H)⁺=236.
Step C:

### Example

### 3-chloro-1-(cyclobutylmethyl)-5-nitro-1H-indazole

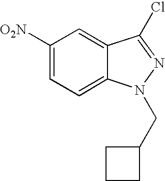 The crude compound obtained following the general procedure step C was purified by flash chromatography (1:3 dichloromethane:toluene) to separate the isomers (desired isomer is less polar). The title compound (3.45 g, 64%) was obtained as a yellow solid. MS (ESI) (M+H)⁺=266.

### Example 13

### N-[3-chloro-1-(cyclobutylmethyl)-1H-indazol-5-yl]butane-1-sulfonamide

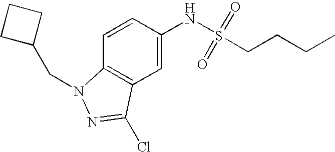 Step A: Following Example 6 step A obtained the desired product. MS (ESI) (M+H)⁺=356.6.
Step B: Example 12 step B
Step C: Example 12 step C

### Example 14

### N-[3-chloro-1-(cyclobutylmethyl)-1H-indazol-5-yl]-2-(methylsulfonyl)benzenesulfonamide

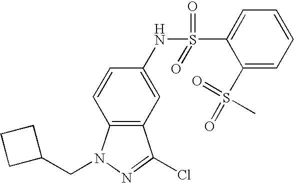 Step A: Following Example 6 step A obtained the desired product. MS (ESI) (M+H)⁺=454.6.
Step B: Example 12 step B
Step C: Example 12 step C

### Example 15

### N-[3-chloro-1-(cyclobutylmethyl)-1H-indazol-5-yl]cyclobutanecarboxamide

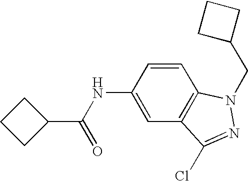 Step A: Following Example 6 step A obtained the desired product. MS (ESI) (M+H)⁺=318.6.
Step B: Example 12 step B
Step C: Example 12 step C

### Example 16

### N-[3-chloro-1-(cyclobutylmethyl)-1H-indazol-5-yl] -2-(2-thienyl)acetamide

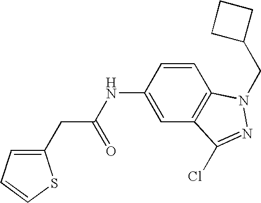 Step A: Following Example 6 step A obtained the desired product. MS (ESI) (M+H)⁺=360.5; t_{R} (min): 1.78.
Step B: Example 12 step B
Step C: Example 12 step C

### Example 17

### N-[1-(4-fluorobenzyl)-1H-indazol-5-yl]-2-(methylsulfonyl)benzenesulfonamide

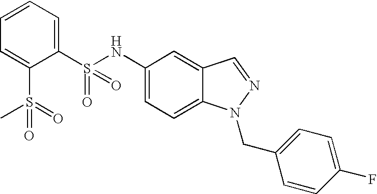 Step A: Following Example 6 step A obtained the desired product. MS (ESI) (M+H)⁺=460.0.
Step B: Example 7 step B
Step C: Example 7 step C

### Example 18

### N-[1-(4-fluorobenzyl)-1H-indazol-5-yl]thiophene-2-carboxamide

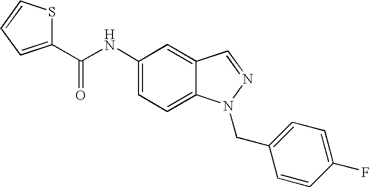 Step A: Following Example 6 step A obtained the desired product. MS (ESI) (M+H)⁺=352.1.
Step B: Example 7 step B
Step C: Example 7 step C

### Example 19

### N′-[3-chloro-1-(cyclobutylmethyl)-1H-indazol-5-yl]-N,N-dimethylsulfamide

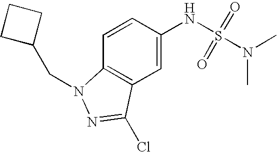 Step A: Following Example 6 step A obtained the desired product. MS (ESI) (M+H)⁺=343.6.
Step B: Example 12 step B
Step C: Example 12 step C

### Example 20

### Procedure (Sulfonamide Formation): Methyl 3-({[1-(4-fluorobenzyl)-1H-indazol-5-yl]amino}sulfonyl)thiophene-2-carboxylate

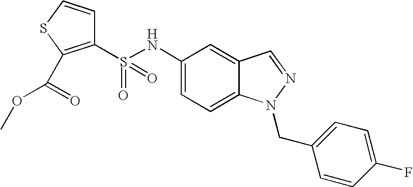 Step A:
To a solution of the free aminoindazole (527 mg, 2.18 mmol, 1 equiv.) and diisopropylethylamine (2 equiv.) in dichloromethane (20 ml) was added the sulfonyl chloride (1.1 equiv.). The reaction mixture was heated at room temperature overnight. Volatiles were evaporated in vacuo. The residue was purified by flash chromatography (silica, hexane/ethyl acetate 2:1) to provide the title compound as the free base (red solid, 715 mg, 1.60 mmol, 74%).
¹H-NMR (CDCl₃): δ ppm 4.03 (s, 3 H) 5.48 (s, 2 H) 6.97 (t, J=8.69 Hz, 2 H) 7.11-7.24 (m, 4 H) 7.35-7.39 (m, 1H) 7.40-7.43 (m, 1H) 7.44 (d, J=0.78 Hz, 1H) 7.94 (d, J=0.78 Hz, 1 H) 8.34 (s, 1H). MS (ESI) (M+H)⁺=446.
Step B: Example 7 step B
Step C: Example 7 step C

### Example 21

### N-[1-(4-fluorobenzyl)-1H-indazol-5-yl]-2-(hydroxymethyl)thiophene-3-sulfonamide

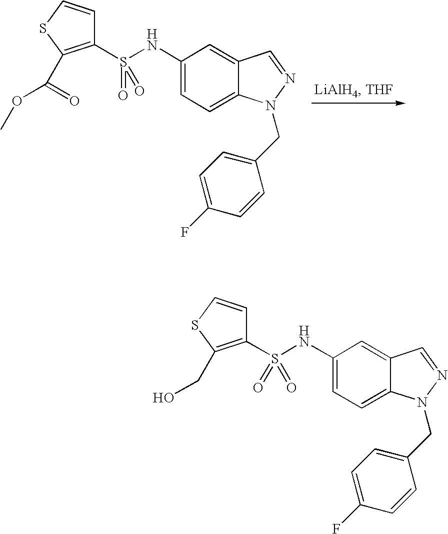 Methyl 3-({[1-(4-fluorobenzyl)-1H-indazol-5-yl] amino}sulfonyl)thiophene-2-carboxylate (55 mg, 0.12 mmol, 1 equiv.) was dissolved in 5 ml of dry THF. Lithium aluminium hydride (0.74 ml of a 0.5M solution in THF, 3 equiv.) was added dropwise. The mixture was stirred at room temperature for 3 days. The reaction was quenched with HCl 2M, then neutralized with a satured NaHCO₃ aquoeus solution. Phases were separated, the aqueous layer was extracted with two portions of ethyl acetate, organic phases were reunited, dried over magnesium sulfate and evaporated in vacuo. The residue was purified by reverse phase HPLC (gradient 10-90% CH₃CN in H₂O) to provide the TFA salt of the title compound after lyophilysation. The title compound (26 mg, 40%) was obtained as a white solid. ¹H-NMR (CDCl₃): δ ppm 4.73 (s, 2 H) 5.54 (s, 2 H) 5.63 (s, 1H) 6.82 (br. s, 1H) 6.99 (t, J=8.69 Hz, 2 H) 7.08-7.20 (m, 5 H) 7.23-7.27 (m, 1H) 7.43 (dd, J=1.95, 0.59 Hz, 1H) 8.01 (dd, J=5.18, 0.90 Hz, 1H). MS (ESI) (M+H)⁺=418.

The following exemplify some of the compounds of the present invention that were made according to the schemes and methods described above. These compounds were found to be active towards the human CB₁ receptor based on the test results of using one or more assays described above.

| | | Observed | t_{R} | |
|---|---|---|---|---|
| Example No. | Structure | MH⁺ | (min) | k′ |
| 22 | 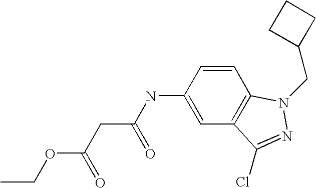 | 350 | 1.62 | |
| 23 | 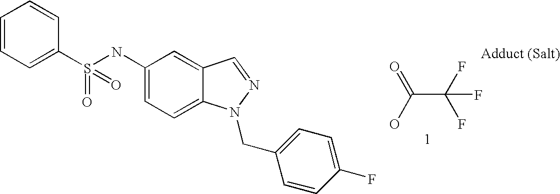 | 382 | 1.69 | |
| 24 | 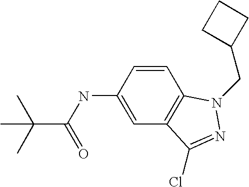 | 320 | 1.78 | |
| 25 | 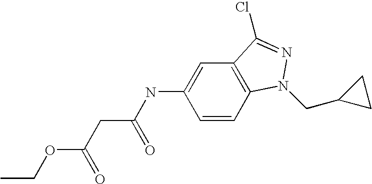 | 336 | 1.51 | |
| 26 | 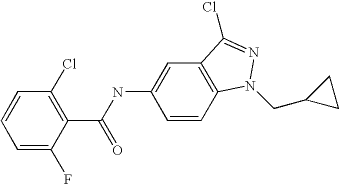 | 378 | 1.76 | |
| 27 | 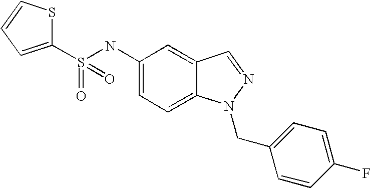 | 388 | 1.64 | |
| 28 | 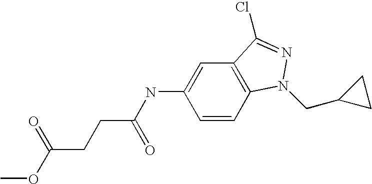 | 336 | 1.44 | |
| 29 | 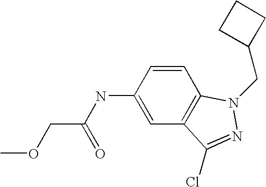 | 308 | 1.53 | |
| 30 | 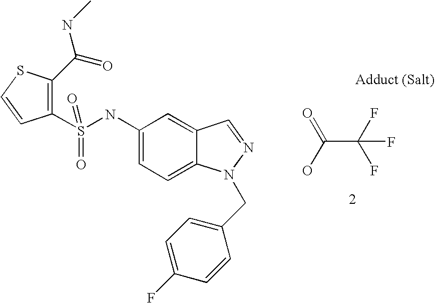 | 445.0 | | 4.93 |
| 31 | 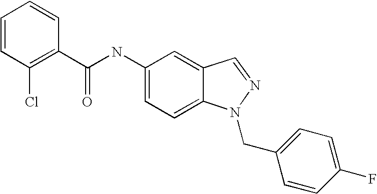 | 380 | 1.68 | |
| 32 | 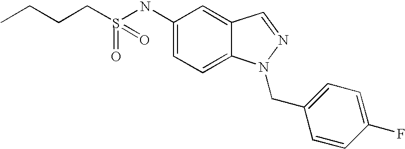 | 362 | 1.66 | |
| 33 | 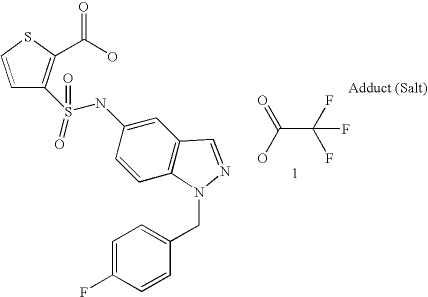 | 432.0 | | 4.78 |
| 34 | 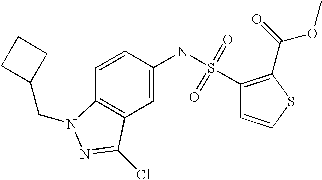 | 440 | 1.89 | |
| 35 | 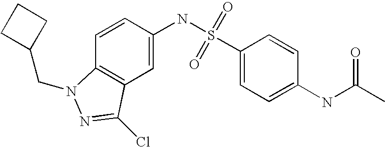 | 433 | 1.61 | |
| 36 | 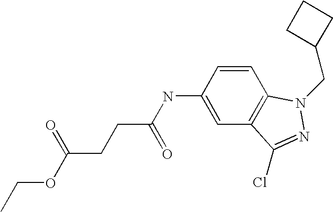 | 364 | 1.64 | |
| 37 | 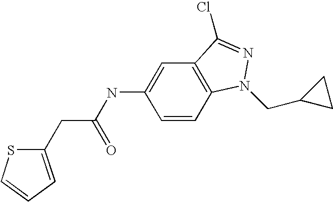 | 346 | 1.69 | |
| 38 | 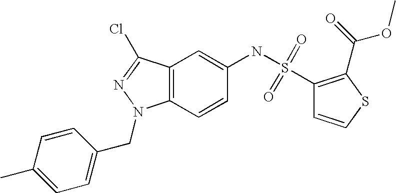 | 476 | 1.93 | |
| 39 | 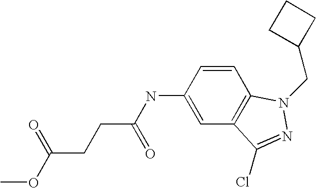 | 350 | 1.56 | |
| 40 | 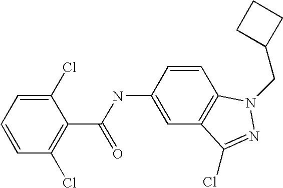 | 408 | 1.91 | |
| 41 | 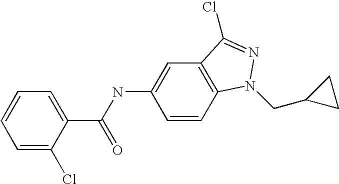 | 360 | 1.76 | |
| 42 | 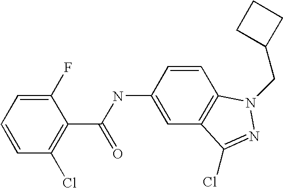 | 392 | 1.86 | |
| 43 | 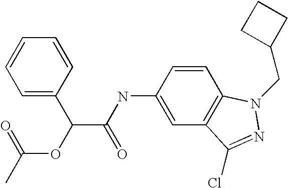 | 412 | 1.83 | |
| 44 | 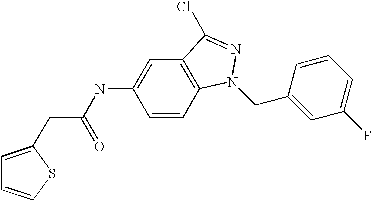 | 400 | 1.81 | |

1. A compound of Formula I or a pharmaceutically acceptable salt thereof: 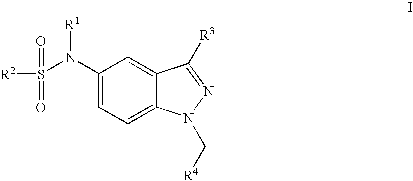
wherein
R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;
R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl used in defining R² is optionally substituted with one or more groups selected from C₁₋₆alkyl, halogen, amino and C₁₋₆alkoxy, 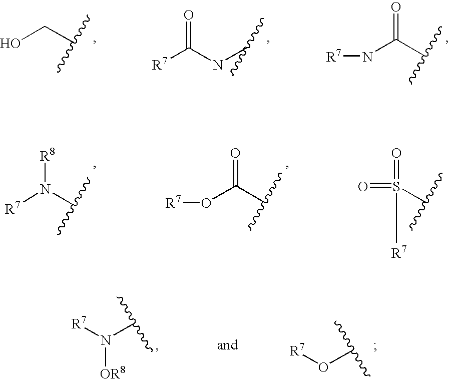
each of R⁷ and R⁸ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆ group that together with another divalent group selected from R⁷ and R⁸ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and divalent C₁₋₆ group used in defining R⁷ and R⁸ is optionally substituted by one or more groups selected from halogen, cyano, nitro, methoxy, ethoxy, methyl, ethyl, hydroxy, and —NR⁵R⁶;
R³ is selected from hydrogen, halogen, amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from C₁₋₄alkyl, C₂₋₄alkenyl, C₂₋₄alkynyl, halogen, C₁₋₄alkoxy, amino, nitro, cyano, oxo, hydroxy, C₁₋₆cycloalkyl-C₁₋₆alkyl, C₃₋₆heterocyclyl, C₃₋₆heterocyclyl-C₁₋₆alkyl, and —NR⁵R⁶
wherein R⁵ and R⁶ are independently selected from —H, C₁₋₈alkyl, C₂₋₈alkenyl, C₂₋₈alkynyl, and a divalent C₁₋₈ group that together with another divalent R⁵ or R⁶ may form a ring or a portion of a ring; and
R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)—; wherein said C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

2. A compound as claimed in claim 1, wherein
R¹ is selected from from —H and C₁₋₆alkyl;
R² is selected from C₁₋₈alkyl, C₂₋₈alkenyl, C₂₋₈alkynyl, C₃₋₈cycloalkyl, C₃₋₈heterocyclyl and C₃₋₈heterocycloalkyl; wherein said C₁₋₈alkyl, C₂₋₈alkenyl, C₂₋₈alkynyl, C₃₋₈cycloalkyl, C₃₋₈heterocyclyl and C₃₋₈heterocycloalkyl used in defining R² is optionally substituted with one or more groups selected from C₁₋₈alkyl, halogen, amino, hydroxy and C₁₋₆alkoxy; 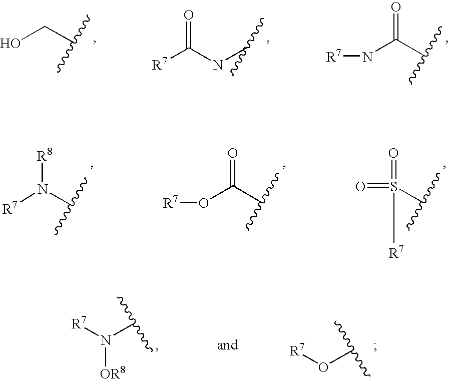
each of R⁷ and R⁸ is independently selected from —H, C₁₋₈alkyl, C₁₋₈alkoxy, C₂₋₈alkenyl, C₃₋₈cycloalkyl, C₃₋₈cycloalkyl-C₁₋₆alkyl, C₃₋₈heterocyclyl and C₃₋₈heterocylcyl-C₁₋₆alkyl; wherein said C₁₋₈alkyl, C₂₋₈alkenyl, C₃₋₈cycloalkyl, C₃₋₈cycloalkyl-C₁₋₆alkyl, C₃₋₈heterocyclyl and C₃₋₈heterocylcyl-C₁₋₆alkyl used in defining R⁷ and R⁸ are optionally substituted by one or more groups selected from halogen, cyano, nitro, methoxy, ethoxy, methyl, ethyl, hydroxy and —NR⁵R⁶;
R³ is selected from hydrogen, halogen, amino, C₁₋₈alkyl, C₂₋₈alkenyl, C₂₋₈alkynyl, C₃₋₈cycloalkyl, C₄₋₈cycloalkenyl, C₃₋₈heteroaryl, R⁵R⁶N—, C₃₋₈cycloalkyl-C₁₋₄alkyl, C₄₋₈cycloalkenyl-C₁₋₄alkyl, phenyl, phenyl-C₁₋₄alkyl, C₃₋₈heterocyclyl or C₃₋₈heterocyclyl-C₁₋₄alkyl; wherein said amino, C₁₋₈alkyl, C₂₋₈alkenyl, C₂₋₈alkynyl, C₃₋₈cycloalkyl, C₄₋₈cycloalkenyl, C₃₋₈heteroaryl, R⁵R⁶N—, C₃₋₈cycloalkyl-C₁₋₄alkyl, C₄₋₈cycloalkenyl-C₁₋₄alkyl, phenyl, phenyl-C₁₋₄alkyl, C₃₋₈heterocyclyl or C₃₋₈heterocyclyl-C₁₋₄alkyl used in defining R³ is optionally substituted by one or more groups selected from halogen, cyano, nitro, C₁₋₄alkoxy, C₁₋₄alkyl, C₂₋₄alkenyl, C₂₋₄alkynyl, hydroxy, and —NR⁵R⁶;
wherein R⁵ and R⁶ are independently selected from from —H, C₁₋₆alkyl, and C₂₋₆alkenyl; and
R⁴ is selected from C₁₋₈alkyl, C₂₋₈alkenyl, C₂₋₈alkynyl, C₃₋₈cycloalkyl, C₃₋₈cycloalkyl-C₁₋₄alkyl, C₄₋₈cycloalkenyl, C₆₋₁₀aryl, phenyl, alkyl, phenyl-C₁₋₄alkyl, C₃₋₈heterocyclyl or C₃₋₈heterocyclyl-C₁₋₄alkyl; wherein said C₁₋₈alkyl, C₂₋₈alkenyl, C₂₋₈alkynyl, C₃₋₈cycloalkyl, C₃₋₈cycloalkyl-C₁₋₄alkyl, C₄₋₈cycloalkenyl, C₆₋₁₀aryl, phenyl, alkyl, phenyl-C₁₋₄alkyl, C₃₋₈heterocyclyl or C₃₋₈heterocyclyl-C₁₋₄alkyl used in defining R⁴ is optionally substituted by one or more groups selected from C₁₋₄alkyl, C₁₋₄alkoxy, halogen, cyano, amino, nitro, oxo, hydroxy, and —NR⁵R⁶.

3. A compound as claimed claim 1, wherein
R¹ is selected from —H and C₁₋₄alkyl;
R² is selected from C₁₋₆alkyl, C₂₋₆alkenyl, C₃₋₆cycloalkyl, C₃₋₆cycloalkenyl, C₃₋₆heterocyclyl, C₃₋₆heterocycloalkyl; wherein said C₁₋₆alkyl, C₂₋₆alkenyl, C₃₋₆cycloalkyl, C₃₋₆cycloalkenyl, C₃₋₆heterocyclyl, C₃₋₆heterocycloalkyl used in defining R² is optionally substituted with one or more C₁₋₆alkyl, C₁₋₆alkoxy, 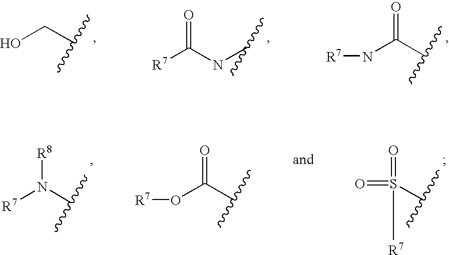
wherein each of R⁷ and R⁸ is independently selected from —H, C₁₋₆alkyl, C₂₋₆alkenyl, C₂₋₆alkynyl, C₁₋₆alkoxy and C₃₋₆cycloalkyl;
R³ is selected from hydrogen, halogen, amino, C₁₋₆alkyl, C₂₋₆alkenyl, C₂₋₆alkynyl, C₃₋₆cycloalkyl, C₃₋₆cycloalkyl-C₁₋₄alkyl, C₃₋₆heterocyclyl or C₃₋₆heterocyclyl-C₁₋₄alkyl wherein said amino, C₁₋₆alkyl, C₂₋₆alkenyl, C₃₋₆cycloalkyl, C₃₋₆cycloalkyl-C₁₋₄alkyl, C₃₋₆heterocyclyl or C₃₋₆heterocyclyl-C₁₋₄alkyl used in defining R³ is optionally substituted by one or more groups selected from Cl, F, methoxy, ethoxy, methyl, ethyl and hydroxy;
R⁵ and R⁶ are independently selected from —H and C₁₃alkyl; and
R⁴ is selected from C₁₋₆alkyl, C₂₋₆alkenyl, C₂₋₆alkynyl, phenyl, alkyl, C₃₋₆cycloalkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, wherein said phenyl, alkyl, phenyl-C₁₋₄alkyl, C₃₋₆cycloalkyl-C₁₋₄alkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, used in defining R⁴ is optionally substituted by one or more groups selected from C₁₋₄alkyl, C₁₋₄alkoxy, halogen, amino, cyano, oxo, hydroxy, and —NR⁵R⁶.

4. A compound as claimed in claim 1, wherein
R¹ is selected from —H and C₁₋₃alkyl;
R² is methyl, ethyl, propyl, t-butyl, n-butyl, phenyl, benzyl, and thienyl, wherein said phenyl, benzyl, and thienyl used in defining R² is optionally substituted with one or more methyl, ethyl, propyl, hydroxy, methoxy, ethoxy, 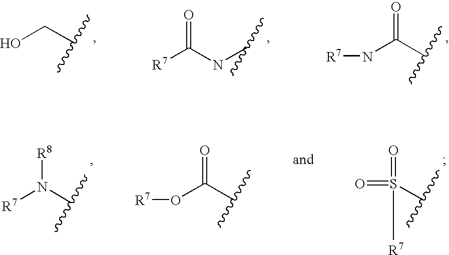
each of R⁷ and R⁸ is independently is selected from —H, methyl, ethyl, propyl, butyl, hydroxy, methoxy;
R³ is selected from hydrogen and Cl; and
R⁴ is selected from C₁₋₄alkyl, C₂₋₄alkenyl, C₂₋₄alkynyl, phenyl, cyclohexyl, cyclopentyl, cyclobutyl, cyclopropyl, cyclohexyl, cyclopentyl, and benzyl; wherein said phenyl, cyclohexyl, cyclopentyl, cyclobutyl, cyclopropyl, cyclohexyl, cyclopentyl, and benzyl used in defining R⁴ is optionally substituted by one or more groups selected from fluorine, chlorine, methoxy, ethoxy, methyl, butyl, propyl, ethyl and hydroxy.

5. A compound of Formula II or a pharmaceutically acceptable salt thereof: 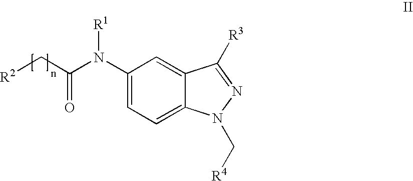
wherein
R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;
R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl, 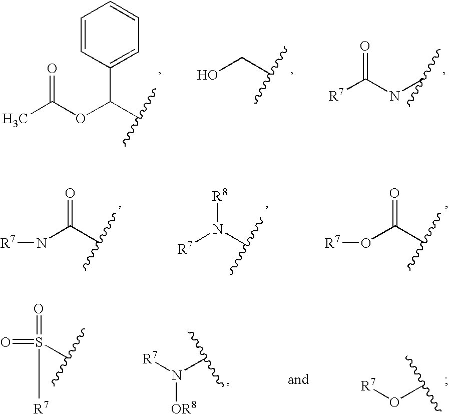
wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl 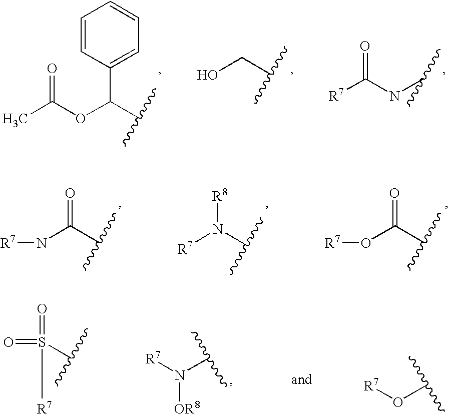
in defining R² is optionally substituted with one or more groups selected from C₁₋₆alkyl, halogen, amino and C₁₋₆alkoxy, 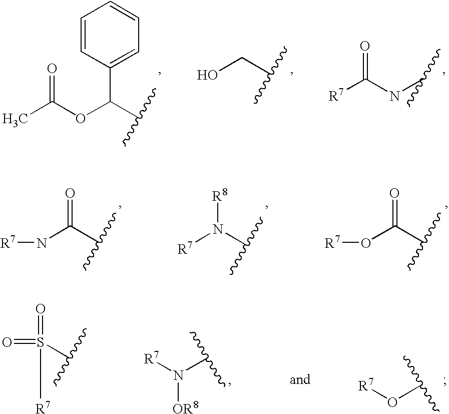
each of R⁷ and R³ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆ group that together with another divalent group selected from R⁸ and R⁹ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, or divalent C₁₋₆ group is optionally substituted by one or more groups selected from halogen, cyano, nitro, methoxy, ethoxy, methyl, ethyl, hydroxy, and —NR⁵R⁶;
n is selected from 0, 1, 2, 3, 4, 5 and 6;
R³ is selected from hydrogen, halogen, amino, C₁₋₁₀alkyl, C₁₋₁₀alkyl-C₃₋₁₀cycloalkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from halogen, cyano, nitro, methoxy, ethoxy, methyl, ethyl, hydroxy, and —NR⁵R⁶;
wherein R⁵ and R⁵ are independently selected from —H, C₁₋₆alkyl, C₂₋₆alkenyl, C₂₋₆alkynyl, and a divalent C₁₋₆ group that together with another divalent R⁵ or R⁶ may form a ring or a portion of a ring; and
R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)—; wherein said C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

6. A compound as claimed in claim 5, wherein
R¹ is selected from —H and C₁₋₆alkyl;
R² is selected from C₁₋₈alkyl, C₂₋₈alkenyl, C₂₋₈alkynyl, C₃₋₈cycloalkyl, C₃₋₈cycloalkyl-C₁₋₄alkyl, C₃₋₆cycloalkenyl, C₃₋₆cycloalkenyl-C₁₋₄alkyl, C₃₋₆heterocyclyl, C₃₋₆heterocycloalkyl, 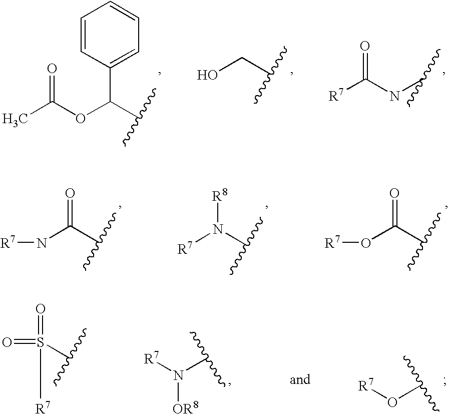
wherein said C₁₋₈alkyl, C₂₋₈alkenyl, C₂₋₈alkynyl, C₃₋₈cycloalkyl, C₃₋₈cycloalkyl-C₁₋₄alkyl, C₄₋₆cycloalkenyl, C₄₋₆cycloalkenyl-C₁₋₄alkyl, C₃₋₆heterocyclyl, C₃₋₆heterocycloalkyl 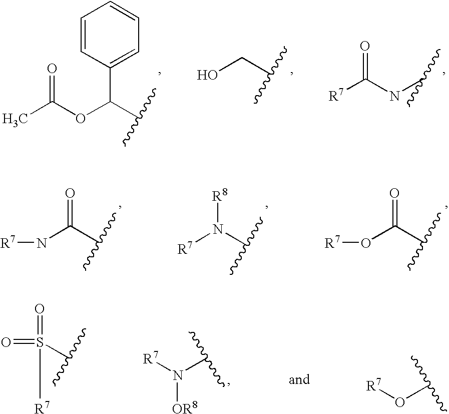
in defining R² is optionally substituted with one or more groups selected from C₁₋₆alkyl, halogen, amino and C₁₋₆alkoxy, 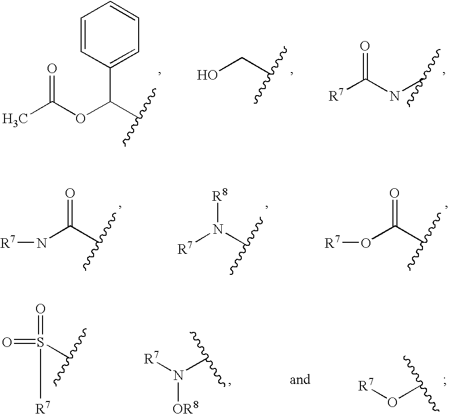
each of R⁷ and R⁸ is independently selected from —H, C₁₋₆alkyl, C₁₋₆alkoxy, C₂₋₆alkenyl, C₃₋₆cycloalkyl, C₃₋₆cycloalkyl-C₁₋₆alkyl, C₃₋₆heterocyclyl and C₃₋₆heterocylcyl-C₁₋₆alkyl, wherein said C₁₋₆alkyl, C₂₋₆alkenyl, C₃₋₆cycloalkyl, C₃₋₆cycloalkyl-C₁₋₆alkyl, C₃₋₆heterocyclyl and C₃₋₆heterocylcyl-C₁₋₆alkyl used in defining R⁷ and R³ are optionally substituted by one or more groups selected from halogen, cyano, nitro, methoxy, ethoxy, methyl, ethyl, hydroxy and —NR⁵R⁶;
n is selected from 0, 1, 2, 3 and 4;
R³ is selected from hydrogen, halogen, amino, C₁₋₈alkyl, C₂₋₈alkenyl, C₂₋₈alkynyl, C₃₋₈cycloalkyl, C₄₋₈cycloalkenyl, C₃₋₅heteroaryl, R⁵R⁶N—, C₃₋₆cycloalkyl-C₁₋₄alkyl, C₄₋₆cycloalkenyl-C₁₋₄alkyl, phenyl, phenyl-C₁₋₄alkyl, C₃₋₆heterocyclyl or C₃₋₆heterocyclyl-C₁₋₄alkyl; wherein said amino, C₁₋₆alkyl, C₂₋₆alkenyl, C₂₋₆alkynyl, C₃₋₆cycloalkyl, C₄₋₆cycloalkenyl, C₃₋₅heteroaryl, R⁵R⁶N—, C₃₋₆cycloalkyl-C₁₋₄alkyl, C₄₋₆cycloalkenyl-C₁₋₄alkyl, phenyl, phenyl-C₁₋₄alkyl, C₃₋₆heterocyclyl or C₃₋₆heterocyclyl-C₁₋₄alkyl used in defining R³ is optionally substituted by one or more groups selected from C₁₋₄alkyl, C₂₋₄alkenyl, halogen, C₁₋₄alkoxy, amino, nitro, cyano, oxo, methoxy, ethoxy, methyl, ethyl, hydroxy, C₁₋₆cycloalkyl-C₁₋₆alkyl, C₃₋₆heterocyclyl, C₃₋₆heterocyclyl-C₁₋₆alkyl, and —NR⁵R⁶;
wherein R⁵ and R⁶ are independently selected from from —H and C₁₋₃alkyl; and
R⁴ is selected from C₃₋₆cycloalkyl, C₃₋₆cycloalkyl-C₁₋₄alkyl, C₄₋₆cycloalkenyl, C₆₋₁₀aryl, phenyl, alkyl, phenyl-C₁₋₄alkyl, C₃₋₆heterocyclyl or C₃₋₆heterocyclyl-C₁₋₄alkyl; wherein said C₃₋₆cycloalkyl, C₃₋₆cycloalkyl-C₁₋₄alkyl, C₄₋₆cycloalkenyl, C₆₋₁₀aryl, phenyl, alkyl, phenyl-C₁₋₄alkyl, C₃₋₆heterocyclyl or C₃₋₆heterocyclyl-C₁₋₄alkyl used in defining R⁴ is optionally substituted by one or more groups selected from C₁₋₄alkyl, C₁₋₄alkoxy, halogen, cyano, amino, nitro, oxo, hydroxy, and —NR⁵R⁶.

7. A compound as claimed in claim 5, wherein
R¹ is selected from —H and C₁₋₄alkyl;
R² is selected from C₁₋₆alkyl, C₂₋₆alkenyl, C₂₋₆alkynyl, C₃₋₆cycloalkyl, C₃₋₆cycloalkyl-C₁₋₄alkyl, C₃₋₆cycloalkenyl, C₃₋₆cycloalkenyl-C₁₋₄alkyl, C₃₋₆heterocyclyl, C₃₋₆heterocycloalkyl, 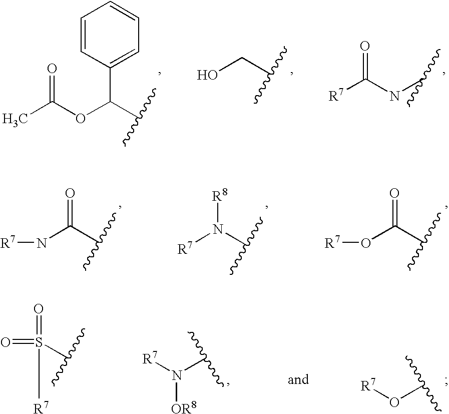
wherein said C₁₋₆alkyl, C₂₋₆alkenyl, C₂₋₆alkynyl, C₃₋₆cycloalkyl, C₃₋₆cycloalkyl-C₁₋₄alkyl, C₃₋₆cycloalkenyl, C₃₋₆cycloalkenyl-C₁₋₄alkyl, C₃₋₆heterocyclyl, C₃₋₆heterocycloalkyl, 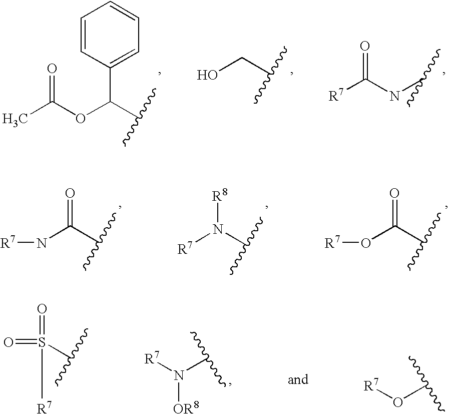
used in defining R² is optionally substituted with one or more groups selected from C₁₋₄alkyl, Cl, F, amino and C₁₋₄alkoxy 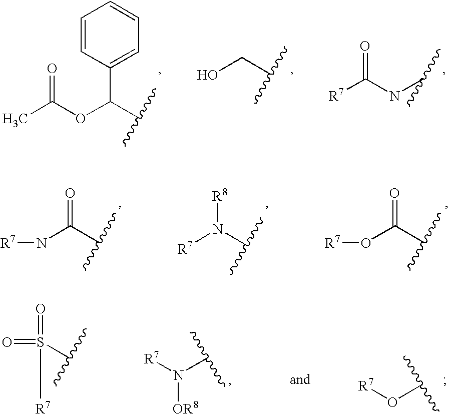
wherein each of R⁷ and R⁸ is independently selected from —H, C₁₋₆alkyl, C₂₋₆alkenyl, C₂₋₆alkynyl, C₁₋₆alkoxy and C₃₋₆cycloalkyl;
n is selected from 0, 1, 2 and 3;
R³ is selected from hydrogen, halogen, amino, C₁₋₆alkyl, C₂₋₆alkenyl, C₂₋₆alkynyl, C₃₋₆cycloalkyl, C₃₋₆cycloalkyl-C₁₋₄alkyl, C₃₋₆heterocyclyl or C₃₋₆heterocyclyl-C₁₋₄alkyl wherein said amino, C₁₋₆alkyl, C₂₋₆alkenyl, C₃₋₆cycloalkyl, C₃₋₆cycloalkyl-C₁₋₄alkyl, C₃₋₆heterocyclyl or C₃₋₆heterocyclyl-C₁₋₄alkyl used in defining R³ is optionally substituted by one or more groups selected from Cl, F, methoxy, ethoxy, methyl, ethyl and hydroxy;
R⁵ and R⁶ are independently selected from —H and C₁₋₃alkyl; and
R⁴ is selected from C₁₋₆alkyl, C₂₋₆alkenyl, C₂₋₆alkynyl, phenyl, alkyl, C₃₋₆cycloalkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, wherein said phenyl, alkyl, phenyl-C₁₋₄alkyl, C₃₋₆cycloalkyl-C₁₋₄alkyl, C₄₋₆cycloalkenyl-C₁₋₄alkyl, C₃₋₆heterocycloalkyl, C₃₋₆heterocycloalkyl-C₁₋₄alkyl, C₆₋₁₀aryl, C₃₋₆cycloalkyl, and C₄₋₆cycloalkenyl, used in defining R⁴ is optionally substituted by one or more groups selected from C₁₋₄alkyl, C₁₋₄alkoxy, halogen, amino, cyano, oxo, hydroxy, and —NR⁵R⁶.

8. A compound as claimed in claim 5, wherein
R¹ is selected from —H and C₁₋₃alkyl;
R² is Cl, F, methyl, ethyl, propyl, t-butyl, n-butyl, hydroxy, methoxy, ethoxy, cyclopropyl, cyclobutyl, benzyl, phenyl, thienyl, 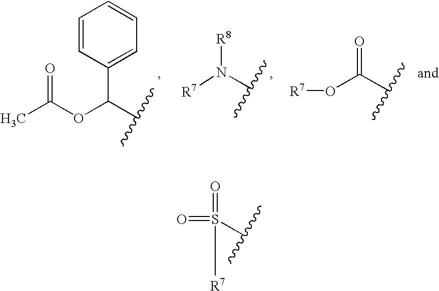
wherein said cyclopropyl, cyclobutyl, benzyl, phenyl, thienyl, 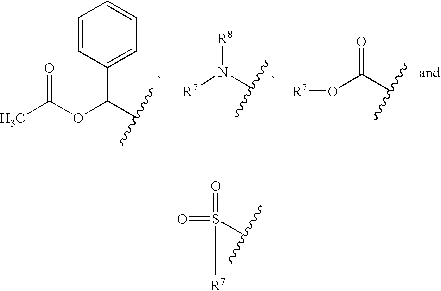
used in defining R² is optionally substituted with one or more Cl, F, C₁₋₄alkyl, 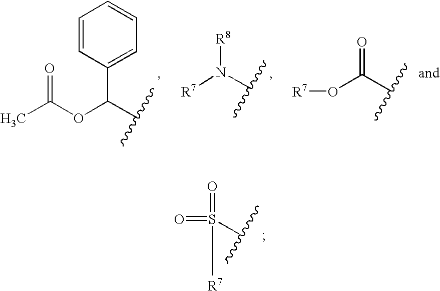
each of R⁷ and R⁸ is independently selected from —H, Cl, F, methyl, ethyl, propyl, t-butyl, n-butyl and C₁₋₄alkoxy;
n is selected from 0, 1 and 2;
R³ is selected from hydrogen and Cl; and
R⁴ is selected from C₁₋₄alkyl, C₂₋₄alkenyl, C₂₋₄alkynyl, phenyl, cyclohexyl, cyclopentyl, cyclobutyl, cyclopropyl, cyclohexyl, cyclopentyl, and benzyl; wherein said phenyl, cyclohexyl, cyclopentyl, cyclobutyl, cyclopropyl, cyclohexyl, cyclopentyl, and benzyl used in defining R⁴ is optionally substituted by one or more groups selected from fluorine, chlorine, methoxy, ethoxy, methyl, butyl, propyl, ethyl and hydroxy.

9-10. (canceled)

11. A method for the treatment of anxiety disorders in a warm-blooded animal, comprising the step of administering to said animal in need of such treatment a therapeutically effective amount of a compound according to claim 1.

12. A method for the treatment of cancer, multiple sclerosis, Parkinson's disease, Huntington's chorea, Alzheimer's disease, gastrointestinal disorders and cardiavascular disorders in a warm-blooded animal, comprising the step of administering to said animal in need of such treatment a therapeutically effective amount of a compound according to claim 1.

13. A pharmaceutical composition comprising a compound according to claim 1 and a pharmaceutically acceptable carrier.

14. A method for the therapy of pain in a warm-blooded animal, comprising the step of administering to said animal in need of such therapy a therapeutically effective amount of a compound according claim 1.

15. A method for preparing a compound of Formula IA, 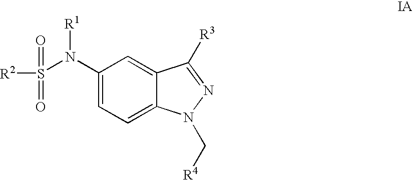
reacting a compound of Formula IB, 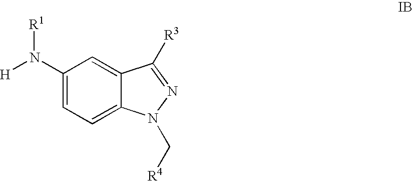
with a compound of R²SO₂Cl in the presence of triethylamine and dichloroethane, wherein
X is selected from 4-fluorophenylmethyl, 2,6-dichlorophenylmethyl, 4-methylphenylmethyl, 3-fluorophenylmethyl, 4-chlorophenylmethyl, 4-methoxyphenylmethyl, 2-fluorophenylmethyl, 2-trifuloromethylphenylmethyl, cyclohexylmethyl, cyclopropylmethyl, cyclobutylmethyl, and alkyl;
R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;
R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, and C₃₋₆heterocycloalkyl used in defining R² is optionally substituted with one or more groups selected from C₁₋₆alkyl, halogen, amino and C₁₋₆alkoxy, 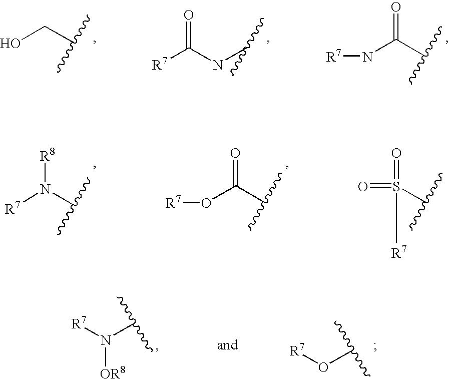
each of R⁷ and R³ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆ group that together with another divalent group selected from R⁷ and R⁸ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and divalent C₁₋₆ group used in defining R⁷ and R⁸ is optionally substituted by one or more groups selected from halogen, cyano, nitro, methoxy, ethoxy, methyl, ethyl, hydroxy, and —NR⁵R⁶;
R³ is selected from hydrogen, halogen, amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from C₁₋₄alkyl, C₂₋₄alkenyl, C₂₋₄alkynyl, halogen, C₁₋₄alkoxy, amino, nitro, cyano, oxo, hydroxy, C₁₋₆cycloalkyl-C₁₋₆alkyl, C₃₋₆heterocyclyl, C₃₋₆heterocyclyl-C₁₋₆alkyl, and —NR⁵R⁶
wherein R⁵ and R⁶ are independently selected from —H, C₁₋₈alkyl, C₂₋₈alkenyl, C₂₋₈alkynyl, and a divalent C₁₋₈ group that together with another divalent R⁵ or R⁶ may form a ring or a portion of a ring; and
R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)—; wherein said C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heteroaryl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, and C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.

16. A method for preparing a compound of Formula IIA, 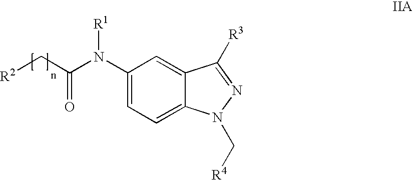
reacting a compound of Formula IIB, 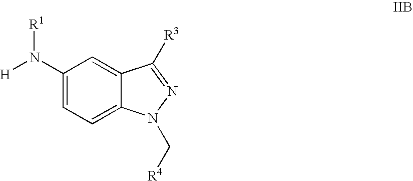
with a compound of R²COCl or R²NCO in the presence of triethylamine and dichloroethane, wherein
X is selected from 4-fluorophenylmethyl, 2,6-dichlorophenylmethyl, 4-methylphenylmethyl, 3-fluorophenylmethyl, 4-chlorophenylmethyl, 4-methoxyphenylmethyl, 2-fluorophenylmethyl, 2-trifuloromethylphenylmethyl, cyclohexylmethyl, cyclopropylmethyl, cyclobutylmethyl, and alkyl;
R¹ is selected from —H, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, and C₄₋₈cycloalkenyl-C₁₋₆alkyl;
R² is selected from C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl, 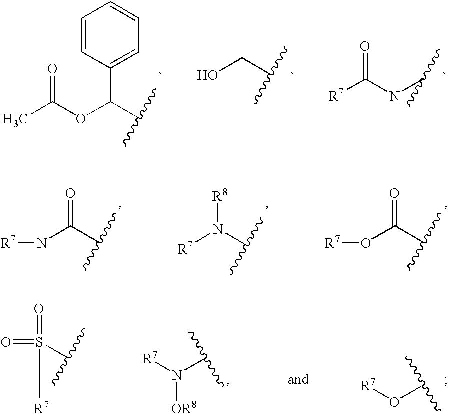
wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl, C₄₋₈cycloalkenyl-C₁₋₆alkyl, C₃₋₆heterocyclyl, C₃₋₆heterocycloalkyl 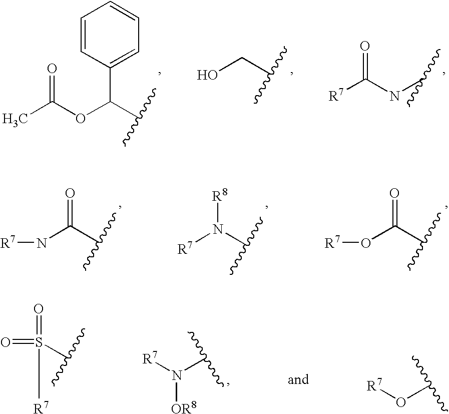
used in defining R² is optionally substituted with one or more groups selected from C₁₋₆alkyl, halogen, amino and C₁₋₆alkoxy, 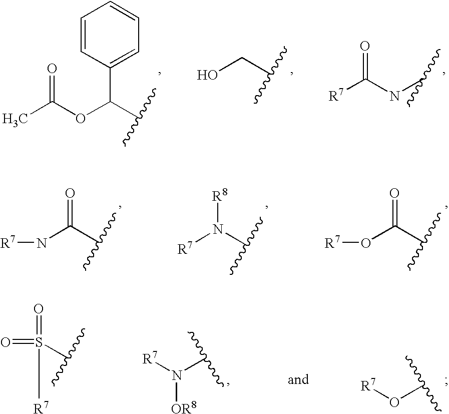
each of R⁷ and R⁸ is independently selected from —H, C₁₋₁₀alkyl, C₁₋₁₀alkoxy, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, and a divalent C₁₋₆ group that together with another divalent group selected from R⁸ and R⁹ forms a portion of a ring, wherein said C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₃₋₆heterocyclyl, C₆₋₁₀aryl, C₃₋₆heterocylcyl-C₁₋₆alkyl, C₆₋₁₀aryl-C₁₋₆alkyl, or divalent C₁₋₆ group is optionally substituted by one or more groups selected from halogen, cyano, nitro, methoxy, ethoxy, methyl, ethyl, hydroxy, and —NR⁵R⁶;
n is selected from 0, 1, 2, 3, 4, 5 and 6;
R³ is selected from hydrogen, halogen, amino, C₁₋₁₀alkyl, C₁₋₁₀alkyl-C₃₋₁₀cycloalkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl, wherein said amino, C₁₋₁₀alkyl, C₂₋₁₀alkenyl, C₂₋₁₀alkynyl, C₃₋₁₀cycloalkyl, C₃₋₁₀cycloalkyl-C₁₋₆alkyl, C₄₋₈cycloalkenyl-C₁₋₆alkyl, C₃₋₆heterocycloalkyl-C₁₋₆alkyl, C₄₋₈cycloalkenyl, R⁵R⁶N—, C₃₋₅heteroaryl, C₆₋₁₀aryl and C₃₋₆heterocycloalkyl used in defining R³ is optionally substituted by one or more groups selected from halogen, cyano, nitro, methoxy, ethoxy, methyl, ethyl, hydroxy, and —NR⁵R⁶;
wherein R⁵ and R⁶ are independently selected from —H, C₁₋₆alkyl, C₂₋₆alkenyl, C₂₋₆alkynyl, and a divalent C₁₋₆ group that together with another divalent R⁵ or R⁵ may form a ring or a portion of a ring; and
R⁴ is selected from C₁₋₁₀alkyl, C₂₋₁₀alkenyl, C₂₋₁₀alkynyl, C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)—; wherein said C₃₋₁₀cycloalkyl, C₄₋₈cycloalkenyl, C₃₋₁₀cycloalkyl-C₁₋₆alkyl, C₄₋₈cycloalkenyl-C₁₋₆alkyl, C₆₋₁₀aryl, C₆₋₁₀aryl-C₁₋₆alkyl, C₃₋₆heterocyclyl, C₃₋₆heterocyclyl-C₁₋₆alkyl, C₆₋₁₀aryl-C(═O)-C₁₋₆alkyl, C₃₋₆heterocyclyl-C(═O)-C₁₋₆alkyl, C₁₋₁₀hydrocarbylamino, C₆₋₁₀aryl-C(═O)—, or C₃₋₆heterocyclyl-C(═O)— used in defining R⁴ is optionally substituted by one or more groups selected from hydrogen, C₁₋₆alkyl, C₂₋₆alkenyl, halogen, C₁₋₆alkoxy, amino, cyano, oxo, nitro, hydroxy and —NR⁵R⁶.
